(19) United States
(12) Patent Application Publication
(10) Pub. No.: US 2024/0044900 A1
(43) Pub. Date: Feb. 8, 2024

(54) FEATURES FOR DETERMINING DUCTAL CARCINOMA IN SITU RECURRENCE AND PROGRESSION

(71) Applicant: The Board of Trustees of the Leland Stanford Junior University, Stanford, CA (US)

(72) Inventors: Tyler Risom, Redwood City, CA (US); Robert B. West, Menlo Park, CA (US); Robert M. Angelo, Stanford, CA (US)

(21) Appl. No.: 18/265,661

(22) PCT Filed: Dec. 10, 2021
(86) PCT No.: PCT/US2021/062909

§ 371 Date: Jun. 6, 2023

Related U.S. Application Data
(60) Provisional application No. 63/123,905, filed on Dec. 10, 2020.

(51) Int. Cl. G01N 33/574; H01J 49/00

(52) U.S. Cl. CPC G01N 33/57415; H01J 49/0004

(57) ABSTRACT

Compositions and methods are provided for stratification of ductal carcinoma in situ (DCIS) tumors with respect to prognostic features that distinguish primary DCIS tumors with a high probability of recurrence and invasive disease, representing tumor progression, from tumors that will not recur. Stratification methods may comprise analysis of a DCIS tissue sample with MIBI-TOF imaging.

### CROSS-REFERENCE TO RELATED APPLICATIONS

This application claims the benefit of PCT Application PCT/US2021/062909, filed Dec. 10, 2021, which claims the benefit of U.S. Provisional Patent Application No. 63/123,905, filed Dec. 10, 2021, which applications are incorporated herein by reference in their entirety.

### GOVERNMENT SUPPORT RESEARCH

This invention was made with Government support under contract CA233254 awarded by the National Institutes of Health. The Government has certain rights in the invention.

### BACKGROUND

Ductal carcinoma in situ (DCIS) is a preinvasive lesion where tumor cells within the breast duct are isolated from the surrounding stroma by a near-continuous layer of myoepithelium and basement membrane proteins. This histologic feature is the central property that distinguishes it from invasive breast cancer (IBC), where this barrier has broken down and tumor cells have invaded the stroma. DCIS comprises 20% of new breast cancer diagnoses, but unlike IBC, in itself is not a life-threatening disease. However, if left untreated, approximately half of these patients will develop IBC within 10 years.

Sequencing-based approaches have been used extensively over the last decade to identify molecular features that could elucidate the connection between DCIS and IBC. Genomic profiling has identified recurrent copy number variants (CNV) that are more prevalent in high-grade DCIS lesions. Meanwhile, comparison of paired DCIS and IBC lesions from the same patient has provided clues into the clonal evolution from in situ to invasive disease by revealing genomic alterations that are acquired during this transition. To date, however, these findings have not been found to consistently explain this transition. Similarly, the utility of tumor phenotyping by single-plex immunohistochemical tissue staining has been limited as well.

In light of this uncertainty, clinical management has trended towards treating all patients presumptively as progressors with surgery, radiation therapy, and pharmacological interventions that carry risks for therapy-related adverse events. Consequently, this approach is likely to be overly aggressive for non-progressors. Thus, understanding the central biological features in DCIS that drive the transition to IBC is a critical unmet need.

Surprisingly, despite all the information now known about the genetic and functional state of tumor cells in DCIS, histopathology remains the only reliable way to diagnose it. Thus, DCIS is an intrinsically structured entity where the spatial orientation of tumor, myoepithelial, and stromal cells is the primary defining feature that distinguishes it from other forms of breast cancer.

### SUMMARY

Compositions and methods are provided for classification of ductal carcinoma in situ (DCIS) lesion with respect to its probability of recurrence and invasive disease. Classification with respect to the probability of cancer recurrence allows treatment appropriate for the condition. While most DCIS is indolent, due to the propensity of some DCIS to become invasive, many subjects with DCIS are treated aggressively. The methods disclosed herein provide a reliable test to determine the propensity of a DCIS lesion to progress to invasive cancer, which allows direction of therapy to those individuals that can benefit from it. Those subjects whose lesions are determined to be indolent can be treated by monitoring the lesion over time, or with low level therapeutics. Those subjects whose lesions have a high probability of invasiveness can receive aggressive therapy, including without limitation surgery, radiation, chemotherapy, immunotherapy, or a combination thereof.

The methods disclosed here utilize a spatial atlas of breast cancer progression identifying features in primary ductal carcinoma in situ (DCIS) that are associated with risk of invasive relapse. Specifically, features related to coordinated transformation of ductal myoepithelium and surrounding stroma are predictive of the clinical outcome. For example, relative to normal tissue, a thin myoepithelial layer in DCIS samples is indicative of whether a patient sample is a DCIS progressor or non-progressor. Analysis of ductal myoepithelium shows that DCIS samples with more continuous myoepithelium and high E-cadherin (ECAD) expression are at higher risk of ipsilateral invasive recurrence following primary DCIS surgical excision. Retention of these normal-like myoepithelial traits correlates with fewer stromal immune cells and cancer associated fibroblasts (CAFs). Conversely, thin, discontinuous, low-ECAD myoepithelium present in non-progressor tumors is correlated with a more reactive desmoplastic stroma with more immune cells, CAFs, and collagen remodeling.

In some embodiments a predictive method is provided for classification of a DCIS tissue from an individual as indolent; or invasive recurrent. The individual may be treated in accordance with the classification. In some embodiments the method comprises analysis of ductal myoepithelium features, where a lesion with myoepitheliem characterized as thin, discontinuous, low-ECAD myoepithelium, relative to a normal control, is classified as indolent. In some embodiments the structure of collagen fibers in the extracellular matrix, and the spatial distribution of multiple immune cell subsets is also analyzed. Imaging of myoepithelium and other features may be performed with multiplexed ion beam imaging by time of flight (MIBI-TOF). The classification can be made by targeted inspection of the imaging data. In some embodiments the method comprises analysis of features extracted from MIBI-TOF data, including, for example, phenotypic, functional, spatial, and morphologic features.

In some embodiments a predictive classifier model is provided for a method for classification of a DCIS tissue from an individual as indolent; or invasive recurrent. In some embodiments the classifier model is a random forest classifier model. In some embodiments a random-forest classifier with MIBI-identified tumor features is trained on patients with known clinical outcomes, and the classifier used to identify those features most useful to separating these outcome groups. The model can be trained to predict recurrence of DCIS and invasive breast cancer (IBC); or can be trained to predict only IBC. In some embodiments the features comprise metrics related to the phenotype of myoepithelium, the structure of collagen fibers in the extracellular matrix, and the spatial distribution of multiple immune cell subsets. The model has identified pixel-level, ECAD⁺ myoepithelial expression as the most predictive metric.

A DCIS sample can be obtained by any means available to those skilled in the art including, but not limited to, a biopsy of the DCIS lesion, including a needle biopsy or surgical removal of tissue containing the lesion. The DCIS lesion can be classified or predicted to be invasive recurrent or indolent based on analysis of the features identified herein. The determination of the aggressiveness phenotype of the DCIS lesion can be used to develop a treatment plan for the subject with the DCIS lesion and to treat the patient accordingly.

In one embodiment, there is provided herein a computer system for determining whether a subject has, is predisposed to having, or has a poor prognosis for, DCIS, comprising: a database of MIBI derived lesion feature datasets, and a server comprising a computer-executable code for causing the computer to receive one or more of the datasets, and to classify the lesion dataset according to a random forest model trained on a dataset of lesion features from tissue with a known outcome, and to generate a classification of whether the lesion is predisposed to invasive, recurrent DCIS. In another aspect, there is provided herein a computer-assisted method for evaluating the prognosis of breast cancer-related disease in a subject, comprising: (1) providing a computer comprising a model or algorithm for classifying data from a DCIS lesion sample obtained from the subject, wherein the classification includes analyzing the data for the presence, absence or amount of MIBI-TOF imaging features (2) inputting data from a biological sample obtained from the subject; and, (3) classifying the biological sample to indicate the DCIS prognosis.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. A longitudinal cohort of DCIS patients with or without subsequent invasive relapse. A. Schematic of the tumor stages and patient sample numbers profiled in this study, including normal breast tissue, primary DCIS, and ipsilateral IBC relapses; 9/12 IBC samples were paired with primary DCIS samples. B. Primary DCIS samples consisted of two outcome groups: progressors, who recurred with ipsilateral invasive disease with a median of 9.1 years, and non-progressors, who never recurred within a median follow-up of 11.4 years.

FIG. 2. A single-cell phenotypic atlas of DCIS epithelium and its microenvironment. A. Depiction of the parallel tissue analysis methods used in this study, including H&E staining, laser- capture microdissection (LCM) of stroma and epithelium with RNAseq, and MIBI-TOF with an overview of the MIBI-TOF workflow. B. Markers used in the MIBI-TOFpanel, grouped by target cell type or protein class. C. Cell lineage assignments based onnormalized expression of lineage markers (heatmap columns). Rows are ordered by absolute abundance (bar plot, left), while columns are hierarchically clustered (euclideandistance, average linkage). Myoep, myoepithelial cell; Mono, monocyte; Endo, endothelial cell; APC, antigen-presenting cell; Macs, macrophages; ImmOther, immune other; MonoDC, monocyte-derived dendritic cell; dnT, double-negative T cell; DC, dendritic cell. D. Representative MIBI image of a DCIS tumor with a 9-color overlay of major cell lineage markers. Inset showing the corresponding H&E image; scale bar=100 μm. Pt., patient. E. A cell phenotype map (CPM) showing cell identity by color, as defined in C, overlaid onto the cell segmentation mask; scale bar=100 μm. F. Region masks marking stroma (pink), myoepithelial (cyan), and ductal (blue) tissue regions; scale bar=100 μm. G. Heatmap of normalized marker expression for four tumor cell subsets including luminal (CK7/PanCK/ECAD+), CK5/7-low (PanCK+, ECAD+only), Basal (CK5/PanCK/ECAD+), and EMT (VIM/PanCK/ECAD+), with an accompanying bar graph of cell subset prominence. H. Images of DCIS tumors with diversity in tumor cell subsets including basal/luminal heterogeneity (left) and EMT tumor cells (right); scale bar=100 μm. I. Heatmap of normalized marker expression for four fibroblast cell subsets including resting fibroblasts (VIM+only, Resting), myofibroblasts (SMA/VIM+, Myo), cancer-associated fibroblasts (FAP/VIM+, CAFs) and normal fibroblasts (CD36/VIM+, Normal). J. Images of DCIS tumors with distinct stroma makeup of fibroblast subsets including normal fibroblast enriched (left) and CAF enriched (right); scale bar=100 μm. K. Area plots of the frequency of tumor subsets (top), fibroblast subsets (middle), and immune lineages (bottom) in all DCIS, IBC, and normal patient samples profiled in this study. Tissue and PAM50 subtype are denoted by color in the top row.

FIG. 3. Transition to DCIS and IBC is marked by coordinated changes in the TME. A. Schematic of the classes of spatial features quantified in all samples, including the measurement of cell type prevalence in specific tissue regions (1: Tissue compartment enrichment), the calculation of paired cell-cell spatial enrichment or spatially enriched cell neighborhoods (2: cell-cell proximity), and morphometric features of the myoepithelial layer and collagen fibers (3: morphometrics). B. Area plot of the distribution of each feature class in the features that significantly differ between normal breast tissue, DCIS, and IBC states by Kruskal-Wallis H test (p<0.05). C. Column plot comparing the prevalence of each feature class in features that differ between tissue states, and total measured features. D. Heatmap of the distinguishing feature prevalence in normal breast tissue, DCIS, and recurrent IBC samples. K-means clustering separated features into four groups of distinct feature-enrichment patterns in the tissues states, including those highest in normal tissue and low in IBC (TME1: Normal Enriched), those highest in DCIS(TME2: DCIS Enriched), and those highest in IBC and low in normal (TME3: IBC Enriched). Features are organized by descending false-discovery rate Q-value within each TME. Color indicates mean over tissue state, z-scored per feature across tissue states. E. Area plot of the distribution of the cellular compartment of the distinguishing features in each TME cluster.

FIG. 4. Increased desmoplasia and ECM remodeling distinguish primary DCIS from their IBC recurrence. A. Paired vertical scatterplot of the stromal density of mast cells in the primary DCIS diagnosis and subsequent IBC recurrence in individual patients; paired Mann-Whitney test. B. The stromal density of normal fibroblasts is compared in longitudinal samples from single patients as in A. C. Representative MIBI image overlays showing the primaryDCIS diagnosis (left) and invasive recurrence (right) from patient 1023. Green arrows, normal fibroblasts, orange arrows, CAFs; scale bar=100 μm. D. Example of dense MIBI collagen signal, collagen fiber object segmentation, and subsequent fiber area and orientation measurement, with fiber-fiber alignment denoted by fiber color. E. Scatter plot comparing summed stromal density of CAFs and myofibroblasts versus collagen fiber density. F. Volcano plot of ECM-related gene expression for the top and bottom CAF-enriched DCIS tumors.

FIG. 5. A. Schematic of the outcome groups of primary DCIS: “progressors,” who recurred with ipsilateral IBC, and “non-progressors,” who showed no recurrence within 11 years of follow-up. MIBI features (N=433) of numerous feature classes were used to train a random forest classifier to differentiate progressor and non-progressor samples. Classifier specificity was then tested on a withheld set of 20% of patients in a test group. B. AUC plot of classifier sensitivity and specificity. C. Classifier accuracy is compared for 10 runs with known progressor/non-progressor labels and 10 runs with randomly permuted progressor/non-progressor labels. P=0.02, Wilcoxon signed rank test. D. Bar plot of features with top classifier importance ranked by average Gini importance across the unpermuted 10 runs. Orange, enriched for progressors; green, enriched for non-progressors. The parent feature class for each feature is shown, and whether that class leveraged spatial information. E. Column plot of the sum of Gini importance of features separated by their corresponding cellular compartment.

FIG. 6. Myoepithelial breakdown and phenotypic change between progressors and non-progressors. A. Representative MIBI image overlay of a DCIS progressor tumor with ECAD co-expression in the SMA+myoepithelium; scale bar=100 μm. B. Boxplot comparing the frequency of ECAD+/SMA+myoepithelial coexpression cluster in progressor (P) and non-progressor (NP) tumors. ***p<0.001, *p<0.05, Mann-Whitney test. C. Boxplot comparing the frequency of the ECAD+myoepithelium in immunofluorescence analysis between P and NP tumors. D. Heatmap of select myoepithelial feature prominence in NP tumors, P tumors, and normal breast tissue. E. Representative images of myoepithelial integrity in normal breast tissue, a P DCIS tumor, and a NP tumor. F. Violin plot of the distribution of linear discriminate analysis-derived “myoepithelial character” values in NP and P tumors as well as normal breast tissue; Kruskal-Wallis test. G. Geneset enrichment analysis of all measured features was used to determine which tissue feature ontologies were enriched in tumors with high or low myoepithelial character scores. Normalized enrichment score is given for each feature ontology; points are colored by significance (false-discovery rate Q-value).

FIG. 7. Representative images of MIBI conjugate staining for all immune markers, with immune control tissues (tonsil, lymph node, and placenta).

FIG. 8. A. Workflow for Deepcell-based segmentation of single cells from multiplexed images. Workflow shows (1) the input data to model training, (2) the model output data of nuclear segmentation, and (3) the multiple sets of parameters used in this study to optimally segment and expand nuclei to identify the diverse cell populations in DCIS. B. Representative image of a DCIS tumor with cell nuclei (gray) shown with cell segmentation outlines (white); scale bar=100 μm.

FIG. 9. A. Schematic of steps involved in single-cell phenotyping, including marker normalization (left), cell clustering into major cellular lineages (middle), and clustering within lineages into cell types (right). B. The major cell subset divisions in each iterative round of phenotype clustering are shown. Cells are first subdivided into cellular lineage, then lineages are further clustered to identify cell types (immune) or phenotypic subsets (tumor, fibroblast). C. Heatmap of the 100 clusters from the round1 lineage clustering. Clusters are annotated by color based on their cell compartment (epithelial: “EPI”, teal; stroma: brown; other: black), as well as their determined final lineage (EPI, green; myoepithelial (“MYOEP”) blue; fibroblast (“FIBRO”) red); endothelial (“ENDO”) brown; immune, gold; other, black. D. Examples of image-based interrogation of cell clusters expressing non-canonical combinations of markers, including a SMA+/CK7+myoepithelial cluster (Cluster 57, top) and a PanCK+/VIM+/CK7-low tumor cluster (12, bottom). E. Heatmap of marker expression in immune lineage cell type clustering, with assigned cell type phenotype to right. F. Heatmap of epithelial marker expression in epithelial lineage cell type clustering. G. Heatmap of clustering in fibroblast lineage.

FIG. 10. A. Representative MIBI image overlays showing an ER₊MER2₋ tumor (left) and ER₋HER2₊ (right), scale bars=100 μm. B. Criteria used to define tumors as ER, AR, HER2, or Ki67 positive, and HER2-intense. C. Area plots showing the frequency of receptor expression states in tumor cells (top), and immune cell type composition (bottom) in all DCIS, IBC, and normal patient samples profiled in this study. Tissue and PAM50 subtype are denoted by color in the top row.

FIG. 11. A. Representative MIBI image overlay of a pure DCIS tumor with major immune cell type markers. Zoomed inset (left) and arrow highlighting intraductal immune phenotypes. Right inset, masked stromal and duct regions where immune cell density is measured. All scale bars=100 μm. B. Heatmap of z-score-normalized cell-type frequency for each cellular neighborhood (CN). C. CN map of the spatial localization of distinct CNs, denoted by color as in B. Insets: Color overlays for lymphocyte-enriched (green dotted line, top) or tumor-interface (red dotted line, bottom) CNs. Scale bar=100 μm. D. Images of SMA signal in normal breast and DCIS with a projected measurement lattice to quantify myoepithelial SMA signal continuity and thickness. Zoomed inset (left) shows myoepithelial SMA signal with nuclear signal (Nuc) and ductal cytokeratin expression (CK); the right inset shows this SMA signal in its binarized form (white) for continuity and thickness measurement. E. Scatterplot of the automated SMA thickness measurement from the method in D compared to SMA thickness measurements made in ImageJ by a blinded pathologist. F. Scatterplot of the automated SMA continuity measurement compared to SMA continuity measurements made in ImageJ by a blinded pathologist. G. Workflow showing the measurement of collagen signal density and collagen fiber morphometrics in three stromal regions (periepithelial, midstroma, distal stroma). Fiber orientation was measured compared to other fibers as well as the epithelial edge. H. Area plot of the distribution of each feature class in all features measured. I. Heatmap of the distinguishing feature prevalence in normal breast, DCIS, and recurrent IBC samples from the TME4: DCIS Low cluster, with all features annotated to the left.

FIG. 12. A. Cell phenotype maps of normal breast tissue, DCIS, and IBC samples showing the distribution of normal fibroblast and CAF states in the stroma, as well as two epithelial states. Insets (left) highlight areas with representative fibroblast makeup with MIBI marker overlays of the same region with fibroblast and epithelial markers shown to the right of the same region. Scale bars=100 μm. B. Boxplot of the quantification of collagen signal in the periepithelial zone of normal breast tissue, DCIS, and IBC samples; p-value from Kruskal-Wallis H test. C. Boxplot of the quantitation of collagen fiber density in the stroma of normal breast tissue, DCIS, and IBC samples; p-value from Kruskal-Wallis H test. D. Boxplot of the quantification of collagen fiber branching in normal breast tissue, DCIS, and IBC samples; p-value from Kruskal-Wallis H test.

FIG. 13. A. Stacked bar plot of the frequency of mastectomy, radiation therapy, and tamoxifen therapy in the progressor (P) and non-progressor (NP) outcome groups in the training data for the recurrence model. B. Distribution of mastectomy, radiation, and tamoxifen therapy is shown by color in the model-predicted progressors (orange) and non-progressors (green), with the random forest prediction probability shown for each patient. P-values comparing the treated frequency of total between groups is displayed, Wilcoxon signed-rank test. C. Stacked column plot of the distribution of spatial versus non-spatial features for all features used in model training (“All”), and those determined to be the 20 most important features by Gini importance test (“Top 20 Gini”). D. Column plot of accumulative Gini importance of features that involve APC cells, dnT cells, or mast cells. E. Column plot of the model's AUC after modifying the correlation cutoff for feature inclusion.

FIG. 14. A. Workflow schematic for pixel-based clustering of myoepithelial phenotype. B. Heatmap of mean marker expression in the seven myoepithelial expression clusters, with a bar plot (left) of cluster abundance out of total identified myoepithelium in the cohort. C. Pseudo-colored image illustrating the spatial distribution of myoepithelial pixel clusters defined in B for a DCIS patient tumor. Scale bars=50 μm. D. Representative immunofluorescent image overlay of DAPI, SMA, and ECAD with zoomed inset of ducts (left) and the myoepithelial objects (right) used to quantify SMA and ECAD coexpression. E. Scatterplot of the quantified myoepithelial ECAD-SMA pixel coexpression by MIBI versus the coexpression quantified in the same patient samples by immunofluorescence.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The types of cancer that can be treated using the subject methods of the present invention include but are not limited to forms of breast cancer, particularly ductal carcinoma in situ. Most breast cancers are epithelial tumors that develop from cells lining ducts or lobules; less common are nonepithelial cancers of the supporting stroma (eg, angiosarcoma, primary stromal sarcomas, phyllodes tumor). Cancers are divided into carcinoma in situ and invasive cancer.

Carcinoma in situ is proliferation of cancer cells within ducts or lobules and without invasion of stromal tissue. There are 2 types: Ductal carcinoma in situ (DCIS): About 85% of carcinoma in situ are this type. DCIS is usually detected only by mammography. It may involve a small or wide area of the breast; if a wide area is involved, microscopic invasive foci may develop over time. Lobular carcinoma in situ (LCIS): LCIS is often multifocal and bilateral. There are 2 types: classic and pleomorphic. Classic LCIS is not malignant but increases risk of developing invasive carcinoma in either breast. This nonpalpable lesion is usually detected via biopsy; it is rarely visualized with mammography. Pleomorphic LCIS behaves more like DCIS; it should be excised to negative margins.

Invasive carcinoma is primarily adenocarcinoma. About 80% is the infiltrating ductal type; most of the remaining cases are infiltrating lobular. Rare types include medullary, mucinous, metaplastic, and tubular carcinomas. Mucinous carcinoma tends to develop in older women and to be slow growing. Women with these rare types of breast cancer have a much better prognosis than women with other types of invasive breast cancer.

Breast cancer invades locally and spreads through the regional lymph nodes, bloodstream, or both. Metastatic breast cancer may affect almost any organ in the body—most commonly, lungs, liver, bone, brain, and skin. Most skin metastases occur near the site of breast surgery; scalp metastases are uncommon. Some breast cancers may recur sooner than others; recurrence can often be predicted based on tumor markers. For example, metastatic breast cancer may occur within 3 years in patients who are negative for tumor markers or occur>10 years after initial diagnosis and treatment in patients who have an estrogen-receptor positive tumor.

When an abnormality is detected during a physical examination or by a screening procedure, testing is required to differentiate benign lesions from cancer. Because early detection and treatment of breast cancer improves prognosis, this differentiation must be conclusive before evaluation is terminated. If advanced cancer is suspected based on physical examination, biopsy should be done first; otherwise, the approach is the same as evaluation for a breast mass, which typically includes ultrasonography. All lesions that could be cancer should be biopsied. A prebiopsy bilateral mammogram may help delineate other areas that should be biopsied and provides a baseline for future reference. However, mammogram results should not alter the decision to do a biopsy if that decision is based on physical findings. Percutaneous core needle biopsy is preferred to surgical biopsy. Core biopsy can be done guided by imaging or palpation (freehand). Routinely, stereotactic biopsy (needle biopsy guided by mammography done in 2 planes and analyzed by computer to produce a 3-dimensional image) or ultrasound-guided biopsy is being used to improve accuracy. Clips are placed at the biopsy site to identify it. If core biopsy is not possible (eg, the lesion is too posterior), surgical biopsy can be done; a guidewire is inserted, using imaging for guidance, to help identify the biopsy site. Any skin taken with the biopsy specimen should be examined because it may show cancer cells in dermal lymphatic vessels. The excised specimen should be x-rayed, and the x-ray should be compared with the prebiopsy mammogram to determine whether all of the lesion has been removed. If the original lesion contained microcalcifications, mammography is repeated when the breast is no longer tender, usually 6 to 12 weeks after biopsy, to check for residual microcalcifications. If radiation therapy is planned, mammography should be done before radiation therapy begins.

Staging follows the TNM (tumor, node, metastasis) classification. Because clinical examination and imaging have poor sensitivity for nodal involvement, staging is refined during surgery, when regional lymph nodes can be evaluated. However, if patients have palpably abnormal axillary nodes, preoperative ultrasonography-guided fine needle aspiration or core biopsy may be done. If biopsy results are positive, axillary lymph node dissection is typically done during the definitive surgical procedure. However, use of neoadjuvant chemotherapy may make sentinel lymph node biopsy possible if chemotherapy changes node status from N1 to N0. (Results of intraoperative frozen section analysis determine whether axillary lymph node dissection will be needed.) If results are negative, a sentinel lymph node biopsy, a less aggressive procedure, may be done instead.

Anatomic Staging of Breast Cancer*

| Stage | Tumor | Regional Lymph Node/Distant Metastasis |
|---|---|---|
| 0 | Tis | N0/M0 |
| IA | T1‡ | N0/M0 |
| IB | T0 | N1mi/M0 |
| | T1‡ | N1mi/M0 |
| IIA | T0 | N1§M0 |
| | T1‡ | N1§/M0 |
| | T2 | N0/M0 |
| IIB | T2 | N1/M0 |
| | T3 | N0/M0 |
| IIIA | TI‡ | N2/M0 |
| | T2 | N2/M0 |
| | T3 | N1/M0 |
| | T3 | N2/M0 |
| IIIB | T4 | N0/M0 |
| | T4 | N1/M0 |
| | T4 | N2/M0 |
| IIIC | Any T | N3/M0 |
| IV | Any T | Any N/M1 |

For most types of breast cancer, treatment involves surgery, radiation therapy, and systemic therapy. Choice of treatment depends on tumor and patient characteristics. Surgery involves mastectomy or breast-conserving surgery plus radiation therapy. Some physicians use preoperative chemotherapy to shrink the tumor before removing it and applying radiation therapy; thus, some patients who might otherwise have required mastectomy can have breast-conserving surgery.

Radiation therapy is indicated after mastectomy if either of the following is present: The primary tumor is ≥5 cm. Axillary nodes are involved. In such cases, radiation therapy after mastectomy significantly reduces incidence of local recurrence on the chest wall and in regional lymph nodes and improves overall survival.

Patients with LCIS are often treated with daily oral tamoxifen. For postmenopausal women, raloxifene or an aromatase inhibitor is an alternative. For patients with invasive cancer, chemotherapy is usually begun soon after surgery. If systemic chemotherapy is not required, hormone therapy is usually begun soon after surgery plus radiation therapy and is continued for years. These therapies delay or prevent recurrence in almost all patients and prolong survival in some. However, some experts believe that these therapies are not necessary for many small (<0.5 to 1 cm) tumors with no lymph node involvement (particularly in postmenopausal patients) because the prognosis is already excellent. If tumors are >5 cm, adjuvant systemic therapy may be started before surgery.

Combination chemotherapy regimens are more effective than a single drug. Dose-dense regimens given for 4 to 6 months are preferred; in dose-dense regimens, the time between doses is shorter than that in standard-dose regimens. There are many regimens; a commonly used one is ACT (doxorubicin plus cyclophosphamide followed by paclitaxel). Acute adverse effects depend on the regimen but usually include nausea, vomiting, mucositis, fatigue, alopecia, myelosuppression, cardiotoxicity, and thrombocytopenia. Growth factors that stimulate bone marrow (eg, filgrastim, pegfilgrastim) are commonly used to reduce risk of fever and infection due to chemotherapy. Long-term adverse effects are infrequent with most regimens; death due to infection or bleeding is rare (<0.2%). High-dose chemotherapy plus bone marrow or stem cell transplantation offers no therapeutic advantage over standard therapy and should not be used.

If tumors overexpress HER2 (HER2+), anti-HER2 drugs (trastuzumab, pertuzumab) may be used. Adding the humanized monoclonal antibody trastuzumab to chemotherapy provides substantial benefit. Trastuzumab is usually continued for a year, although the optimal duration of therapy is unknown. If lymph nodes are involved involvement, adding pertuzumab to trastuzumab improves disease-free survival. A serious potential adverse effect of both these anti-HER2 drugs is a decreased cardiac ejection fraction. With hormone therapy (eg, tamoxifen, raloxifene, aromatase inhibitors), benefit depends on estrogen and progesterone receptor expression; benefit is greatest when tumors have expressed estrogen and progesterone receptors.

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Decrease in survival: As used herein, “decrease in survival” refers to a decrease in the length of time before death of a patient, or an increase in the risk of death for the patient.

Poor prognosis: Generally refers to a decrease in survival, or in other words, an increase in risk of death or a decrease in the time until death. Poor prognosis can also refer to an increase in severity of the disease, such as an increase in spread or invasiveness (metastasis) of the cancer to other tissues and/or organs.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

In some embodiments of the present methods, use of a control is desirable. In that regard, the control may be a non-cancerous tissue sample obtained from the same patient, or a tissue sample obtained from a healthy subject, such as a healthy tissue donor. In another example, the control is a standard calculated from historical values. In one embodiment the control is a cancerous tissue sample of breast cancer. The control may be derived from tissue of known dysplasia, known cancer type, known mutation status, and/or known tumor stage. In one embodiment the control is a historical average derived from DCIS.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa- 2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (Iapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### Methods

Methods are provided for prognostic determination for recurrence of DCIS breast cancer, including recurrence as DCIS or recurrence as IBC, allowing classification of patients based on the determination. Patients can be treated in accordance with the determination, where predicted aggressiveness of a DCIS lesion can be used to develop a treatment plan for the subject with the lesion. It is shown herein that such breast cancer progression is associated with a reduction in myoepithelial integrity, a shift in fibroblast function towards proliferative cancer-associated states (CAFs), and remodeling of collagen in the extracellular matrix (ECM).

In some embodiments a predictive method is provided for classification of a DCIS tissue from an individual as indolent; or invasive recurrent. In some embodiments the method comprises analysis of ductal myoepithelium features, where myoepitheliem characterized as thin, discontinuous, low-ECAD myoepithelium, relative to a normal control, is classified as indolent. In some embodiments the structure of collagen fibers in the extracellular matrix, and the spatial distribution of multiple immune cell subsets is also analyzed. In some embodiments a plurality of features obtained by MIBI-TOF analysis of a DCIS lesion are used for classification.

A DCIS sample can be obtained by any means available to those skilled in the art including, but not limited to, a biopsy of the DCIS lesion, including a needle biopsy or surgical removal of tissue containing the lesion. For example, a tissue slide or block is obtained. The tissue is optionally frozen or fixed. A plurality of tissue samples can be aggregated in a tissue microarray for convenience of analysis, optionally combined with samples of positive and/or negative controls. Serial sections of a slide can be cut for H&E staining to guide imaging, and for MIBI-TOF imaging.

In some embodiments the DCIS sample is stained with a panel of antibodies to define the cellular composition and structural characteristics of the tissue. In some embodiments the antibodies are conjugated directly or indirectly with a detectale marker, e.g. isotopic metal reporters, fluorescent dyes, and the like as known in the art. The slides are contacted with antibodies, usually a panel of antibodies, and then washed free of unbound antibodies.

In some embodiments the panel of antibodies comprises antibodies specific for one or more markers: Tryptase, CK7, VIM, CD44, CK5, PanCK, HIF1A, CD45, AR, HLADR/DP/DQ, GLUT1, ECAD, CD20, MMP9, FAP, CD11c, HER2, CD3, CD8, CD36, MPO, CD68, pS6, Granzyme B, P63, Ki67, IDO1, CD31, PD1, CD14, CD4, Collagen 1, SMA, COX2, Histone H3, ER, PDL1-biotin. In some embodiments the panel comprises at least 5, at least 10, at least 15, at least 20, at least 25, at least 30, at least 35 or all of these markers. In some embodiments a panel of antibodies as defined above comprises at least an antibody specific for E-cadherin.

In some embodiments features are obtained from MIBI-TOF and antibody staining to generate parameters, or features, for classification, where multiplexed image sets are extracted and filtered. Deepcell segmentation parameters are optionally generated. Single cell expression of markers may be measured and normalized.

In some embodiments the features for classification comprise one or more of: myoepithelial E-cadherin expression, antigen presenting cells (APC) near endothelium, periductal immune cells, ER+luminal tumor cells, ER+tumor cells, myoepithelial CK5 expression, tumor-myoepithelium neighborhood, APC near fibroblast, CD8+T cells near double negative T cells (dnT), myoepithelial continuity, CD4+T cells near dnT, stromal mast cells, PDL1+CK5/7-low tumor cells, tumor-dominate neighborhood, B cell near dnT, macrophage near mast cells, CD8+T cells near mast cells, variation in collagen fiber orientation, periductal APCs, PD1+immune cells.

In some embodiments features for classification comprise at least myoepithelial E-cadherin. In some embodiments, features for classification comprise at least each of myoepithelial E-cadherin expression, antigen presenting cells (APC) near endothelium, periductal immune cells, ER+luminal tumor cells, ER+tumor cells, myoepithelial CK5 expression, tumor-myoepithelium neighborhood, APC near fibroblast, CD8+T cells near double negative T cells (dnT), myoepithelial continuity, CD4+T cells near dnT, stromal mast cells, PDL1+CK5/7-low tumor cells, tumor-dominate neighborhood, B cell near dnT, macrophage near mast cells, CD8+T cells near mast cells, variation in collagen fiber orientation, periductal APCs, PD1+immune cells. In some embodiments features for classification include additional features set forth in Table 1, e.g. at least 10, at least 20, at least 30, at least 40, at least 50 or more of the features, and may comprise all of the features set forth in Table 1.

An image of the tissue can be captured, transformed into data, and transmitted to a biological image analyzer for analysis, which biological image analyzer comprises a processor and a memory coupled to the processor, the memory to store computer-executable instructions that, when executed by the processor, cause the processor to perform operations comprising the classification processes disclosed herein. For example, the tissue may be analyzed, digitized, and either stored onto a non-transitory computer readable storage medium or transmitted as data directly to the biological image analyzer for analysis. As another example, a the stained tissue may be scanned, digitized, and either stored onto a non-transitory computer readable storage medium or transmitted as data directly to a computer system for analysis. In one embodiment, features are automatically identified.

In some embodiments, machine learning tools for multiplexed cell segmentation and spatial analytics are used to enumerate cell populations and to quantify how these populations are spatially distributed relative to one another. Object morphometrics and high dimensional pixel clustering are used to annotate the structure of stromal collagen and myoepithelial phenotypes that track with disease progression.

The features quantified in these analyses can be used to build a random forest classifier for predicting which patients will progress to invasive disease based exclusively on the original DCIS biopsy.

In some embodiments a predictive classifier model is provided for a method for classification of a DCIS tissue from an individual as indolent; or invasive recurrent. In some embodiments the classifier model is a random forest classifier model. In some embodiments a random-forest classifier with MIBI-identified tumor features is trained on patients with known clinical outcomes, and the classifier used to identify those features most useful to separating these outcome groups. The model can be trained to predict recurrence of DCIS and invasive breast cancer (IBC); or can be trained to predict only IBC. In some embodiments the features comprise metrics related to the phenotype of myoepithelium, the structure of collagen fibers in the extracellular matrix, and the spatial distribution of multiple immune cell subsets. For example, the model has identified pixel-level, ECAD⁺ myoepithelial expression as the most predictive metric.

### Computer Aspects

A computational system (e.g., a computer) may be used in the methods of the present disclosure to control and/or coordinate stimulus through the one or more controllers, and to analyze data from imaging DCIS samples. A computational unit may include any suitable components to analyze the measured images. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

The raw data from measurements can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems. Such presentation provides a skilled artisan with a ranking of similarities and identifies the degree of similarity contained in the test data.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as drug discovery, analysis of interactions between cellular components, and the like. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein. A variety of structural formats for the input and output means can be used to input and output the information in the computer-based systems of the present invention.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data (e.g., immune repertoire analysis results), can be input into a computer by a user either directly or indirectly. Additionally, any of the devices which can be used to analyze features can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Transition to Invasive Breast Cancer is Associated with Progressive Changes in the Structure and Composition of Tumor Stroma

Ductal carcinoma in situ (DCIS) is a pre-invasive lesion that is thought to be a precursor to invasive breast cancer (IBC). To understand the changes in the tumor microenvironment (TME) accompanying transition to IBC, we used multiplexed ion beam imaging by time of flight (MIBI-TOF) and a 37-plex antibody staining panel to interrogate 79 clinically annotated surgical resections using machine learning tools for cell segmentation, pixel-based clustering, and object morphometrics. Comparison of normal breast with patient-matched DCIS and IBC revealed coordinated transitions between four TME states that were delineated based on the location and function of myoepithelium, fibroblasts, and immune cells. Surprisingly, myoepithelial disruption was more advanced in DCIS patients that did not develop IBC, suggesting this process could be protective against recurrence. Taken together, this HTAN Breast PreCancer Atlas study offers new insight into drivers of IBC relapse and emphasizes the importance of the TME inregulating these processes.

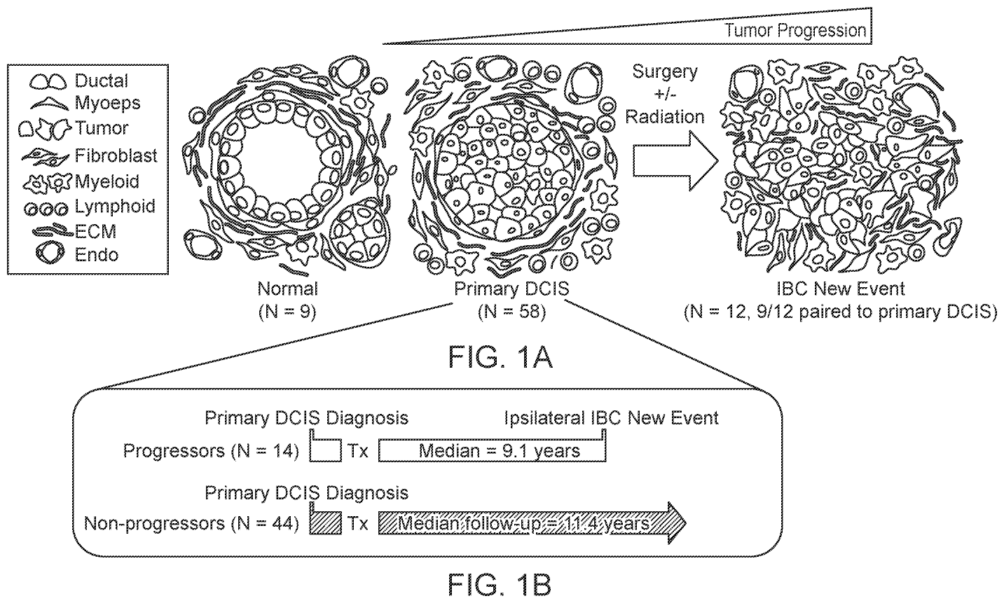

Ductal carcinoma in situ (DCIS) is a pre-invasive lesion of tumor cells within the breast duct that are isolated from the surrounding stroma by a near-continuous layer of myoepithelium and basement membrane proteins. This histologic property is the primary feature that distinguishes DCIS from invasive breast cancer (IBC), where this barrier is absent and tumor cells are in direct contact with the stroma (FIG. 1A). DCIS comprises 20% of new breast cancer diagnoses, but unlike IBC, is not a life-threatening disease in itself. However, if left untreated, up to half of patients with DCIS develop IBC within 10 years, leading to the current practice of surgical intervention for all DCIS patients.

Sequencing-based approaches have been used extensively over the last decade to identify molecular mechanisms that could explain the connection between DCIS and IBC. Genomic profiling has identified recurrent copy number variants that are more prevalent in high-grade DCIS lesions. Comparison of DCIS and IBC lesions from the same patient has provided clues into the clonal evolution from in situ to invasive disease by revealing genomic alterations that are acquired during this transition. To date, however, these findings have not consistently explained this transition. Similarly, the utility of tumor phenotyping by single-plex immunohistochemical tissue staining has been limited as well.

In light of this uncertainty, clinical management has favored treating all patients presumptively as progressors to IBC with surgery, radiation therapy, and pharmacological interventions, all of which carry risks for adverse events. Consequently, this approach is likely to be overly aggressive for patients who do not progress (non-progressors). Thus, understanding what drives DCIS to transition to IBC is a critical unmet need and opportunity for prevention. Surprisingly, despite all the information now known about the genetic and functional state of tumor cells in DCIS, histopathology remains the only reliable way to diagnose it. Thus, DCIS is an intrinsically structured entity for which the spatial orientation of tumor, myoepithelial, and stromal cells are defining characteristics.

To understand how DCIS structure and single-cell function are interrelated, we used new tools previously developed by our lab for highly multiplexed subcellular imaging to analyze a large cohort of human archival tissue samples covering the spectrum of breast cancer progression from in situ to invasive disease in a spatially resolved manner. In previous work, we used MIBI-TOF to identify rule sets governing the tumor microenvironment (TME) structure in triple-negative breast cancer that were highly predictive of the composition of immune infiltrates, the expression of immune checkpoint drug targets, and 10-year overall survival. This effort provided a framework for how TME structure and composition could be used more generally as a surrogate readout to understand the functional response to neoplasia. With this in mind, we sought to determine to the extent to which similar themes involving myoepithelial, stromal, and immune cells in the DCIS TME might play pivotal roles in breast cancer progression. These cell types have been implicated previously in promoting local invasion, metastasis, and correlation with clinical progression.

Here, we report the first systematic, high-dimensional analysis of breast cancer progression using the Washington University Resource Archival Human Breast Tissue (RAHBT) cohort, a clinically annotated set of archived tissue from patients diagnosed with DCIS and IBC. Because the DCIS patient population is complicated by differences in age, parity status, tumor subtype, and treatment course, a well-conceived cohort design is crucial for identifying meaningful features amidst these confounding variables. The RAHBT cohort was therefore composed of primary DCIS tumors from women who later progressed to IBC that were matched by age and year of diagnosis with DCIS from women who did not have a subsequent ipsilateral breast event. We used MIBI-TOF and a 37-plex antibody staining panel to comprehensively define the cellular composition and structural characteristics in normal breast tissue, DCIS, and IBC relapses. These findings were corroborated by transcriptomic data acquired from adjacent co-registered tissue regions isolated by laser capture microdissection. We used the 433 parameters quantified in these analyses to build a random forest classifier for predicting which DCIS patients would later progress to IBC based on the original resection specimen. This classifier was heavily weighted for spatially informed parameters quantifying breast cancer TME structure, particularly those relating to ductal myoepithelium. Surprisingly, myoepithelial loss was more pronounced in samples from DCIS patients that did not recur and was typically associated with a more reactive stroma. Taken together, the studies reported here provide new insight into potential etiologies of DCIS progression that will guide development of future diagnostics and serve as a template for how to conduct similar analyses of pre-invasive cancers.

Results

A longitudinal cohort of DCIS patients with or without subsequent invasive relapse. The goal of this study was to explore two central questions of breast cancer progression. First, how does the structure, composition, and function of breast tissue change with progression from DCIS to IBC? Second, what distinguishes DCIS lesions in patients that later develop IBC (progressors) from those that do not (non-progressors)? To examine these questions, we mapped the phenotype, structure, and spatial distribution of tumor, myoepithelium, stroma, and immune cells of 79 archival formalin-fixed paraffin-embedded patient tissues from the RAHBT cohort (FIG. 1A)

Patient samples included normal breast tissue (N=9, reduction mammoplasty), primary DCIS (N=58), and IBC (N=12). Of the 58 primary DCIS samples, 44 were from non-progressors (median follow-up=11.4 years), while the remaining 14 were from progressors (median time to subsequent breast event=9.1 years, FIG. 1B). Importantly, all IBC tissues were ipsilateral breast events from patients with a prior diagnosis of DCIS, 9/12 of which were longitudinal samples that were matched to a progressor DCIS sample.

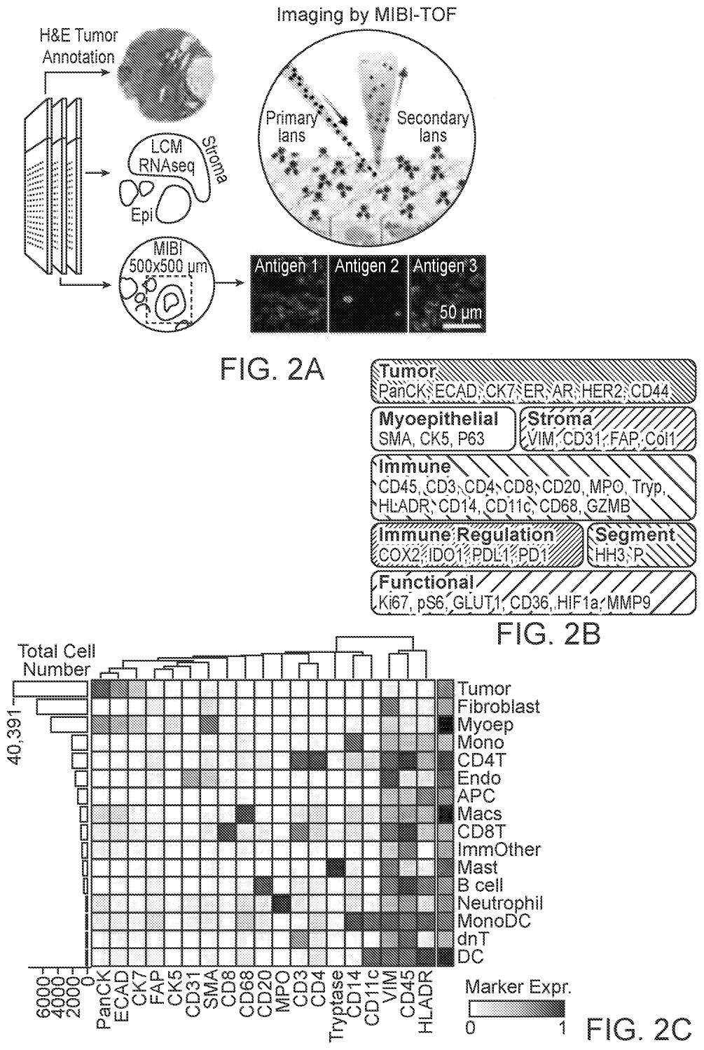

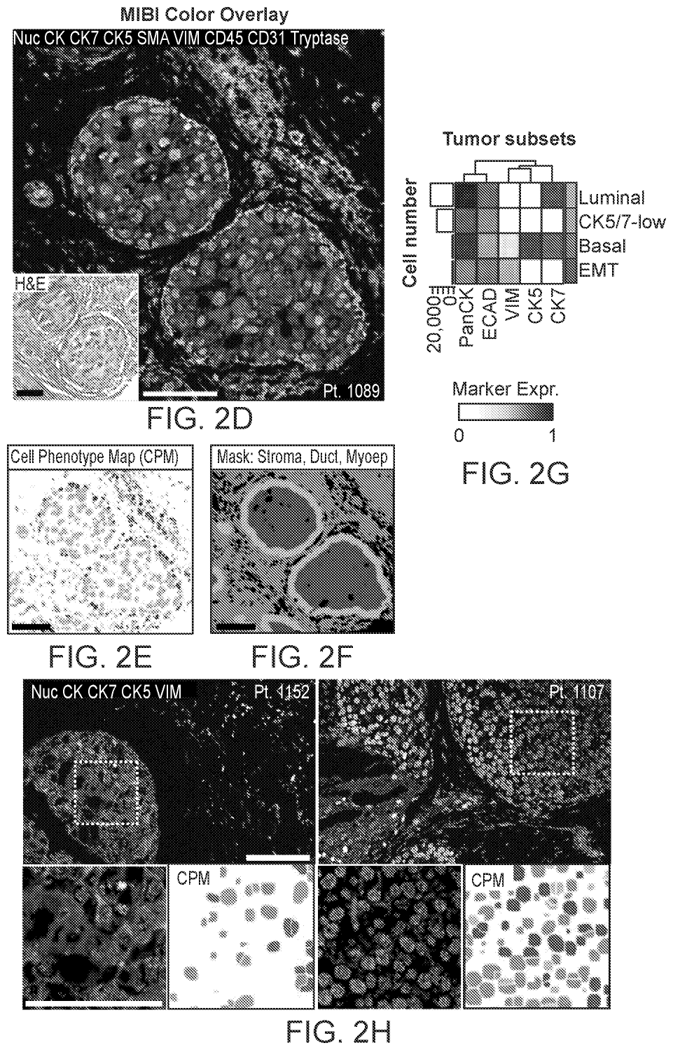

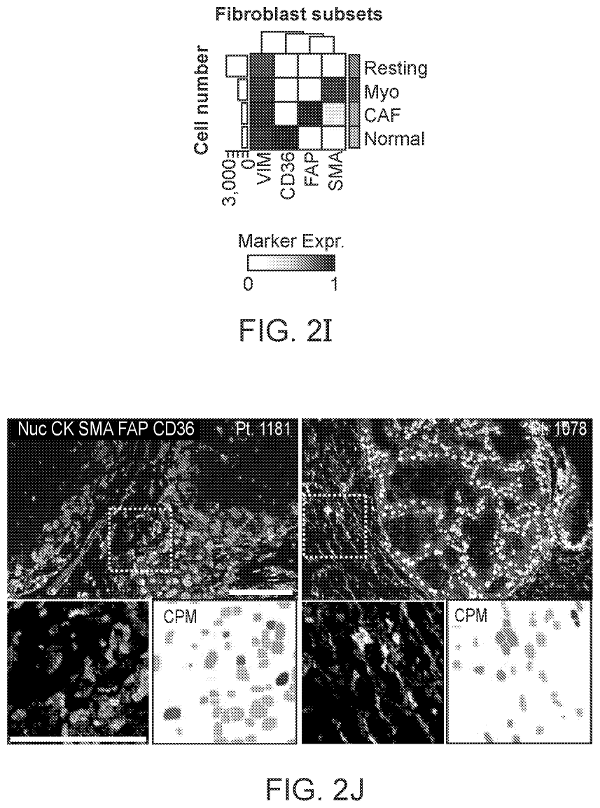

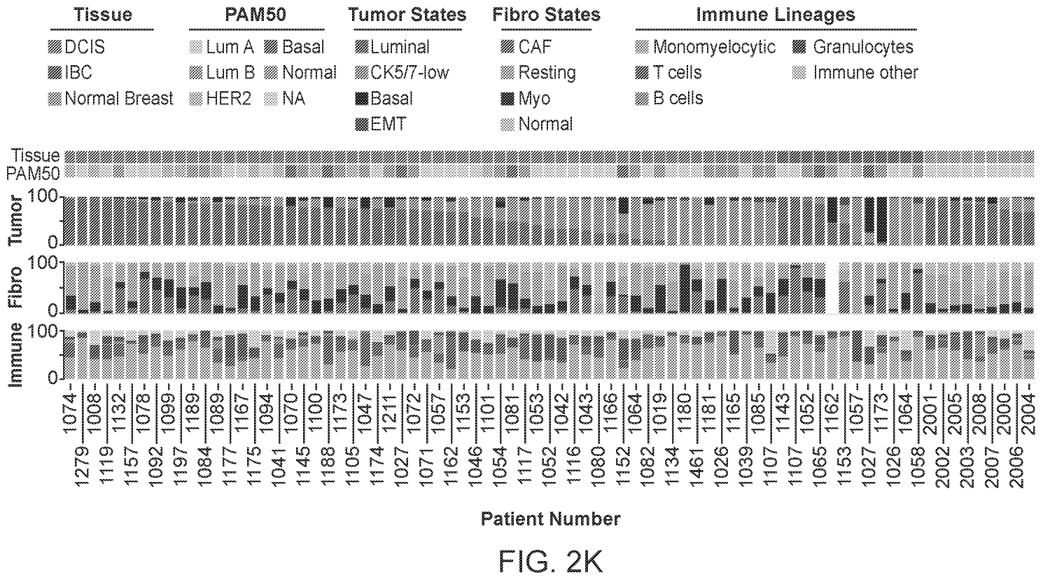

A single-cell phenotypic atlas of DCIS epithelium and its microenvironment. As part of the HTAN PreCancer Atlas, we created a multiomic atlas of breast cancer progression using co-registered adjacent serial sections cut from each RAHBT tissue microarray (TMA) block. For this study, these tissues were used for hematoxylin and eosin (H&E) histochemical staining, RNA transcriptome laser-capture microdissection (LCM-Smart-3SEQ), and highly multiplexed imaging (MIBI-TOF, FIG. 2A). The location of DCIS-containing ducts in H&E sections were manually demarcated by a breast pathologist. This information was then used to guide spatial co-registration of LCM-Smart-3SEQ and MIBI-TOF analyses to ensure that the same ductal and stromal regions were sampled with each technique.

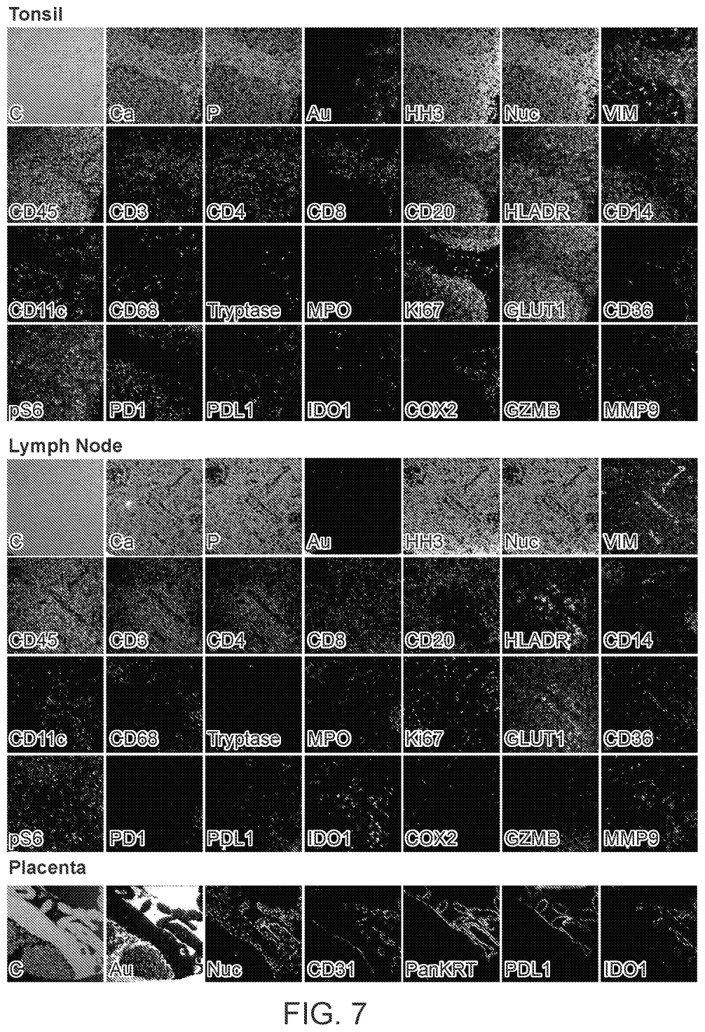

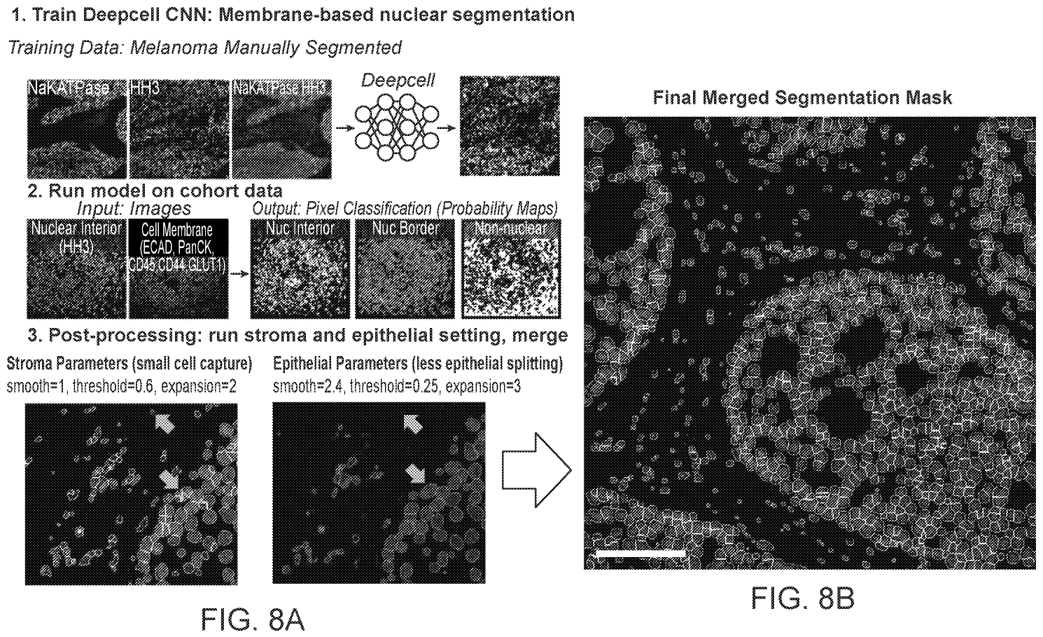

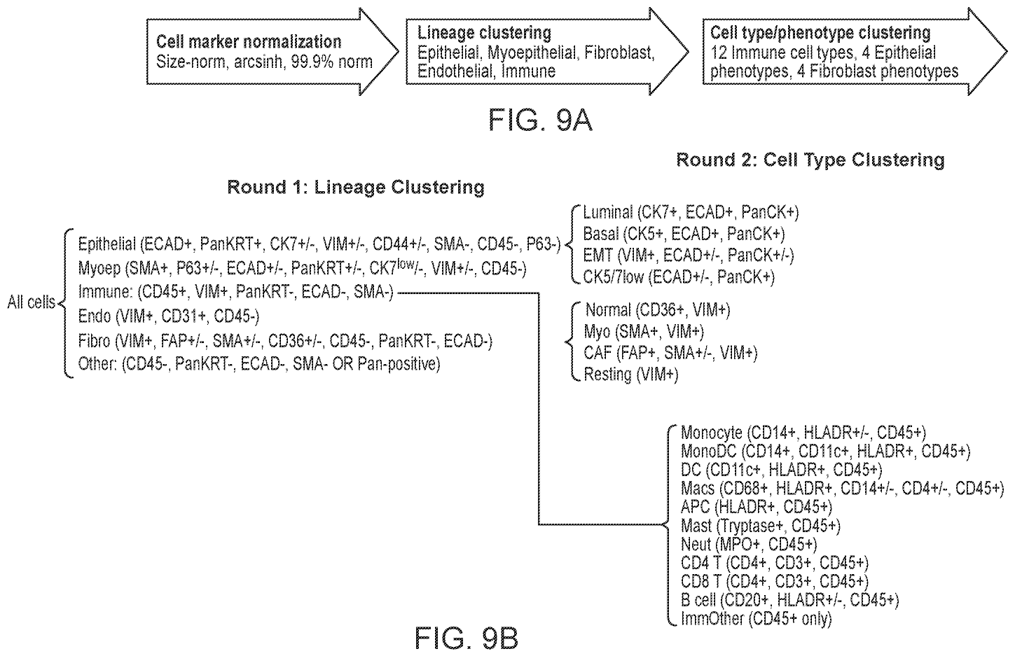

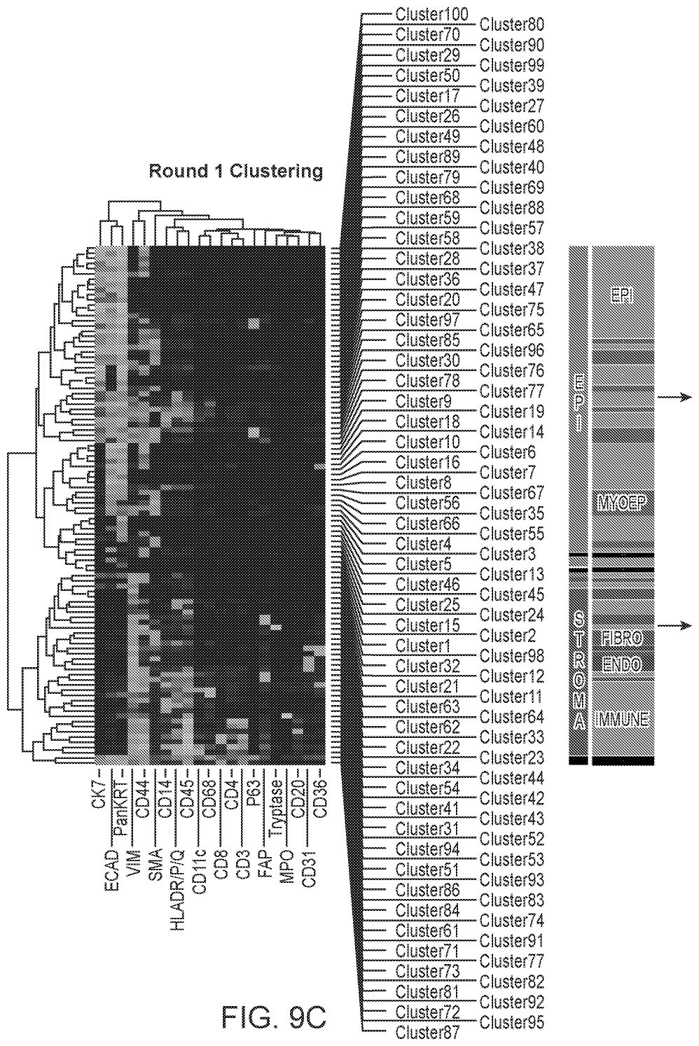

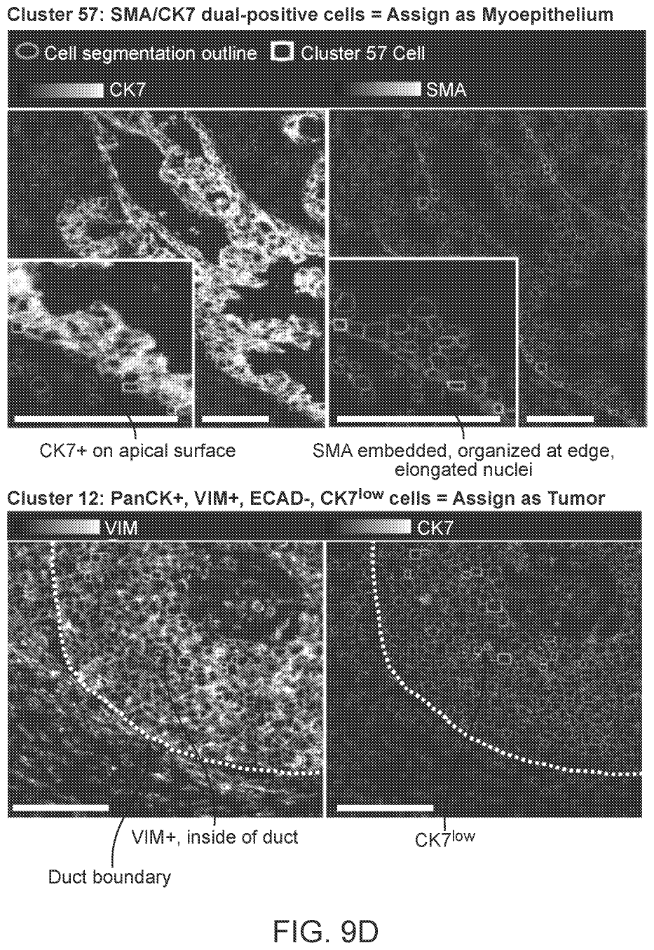

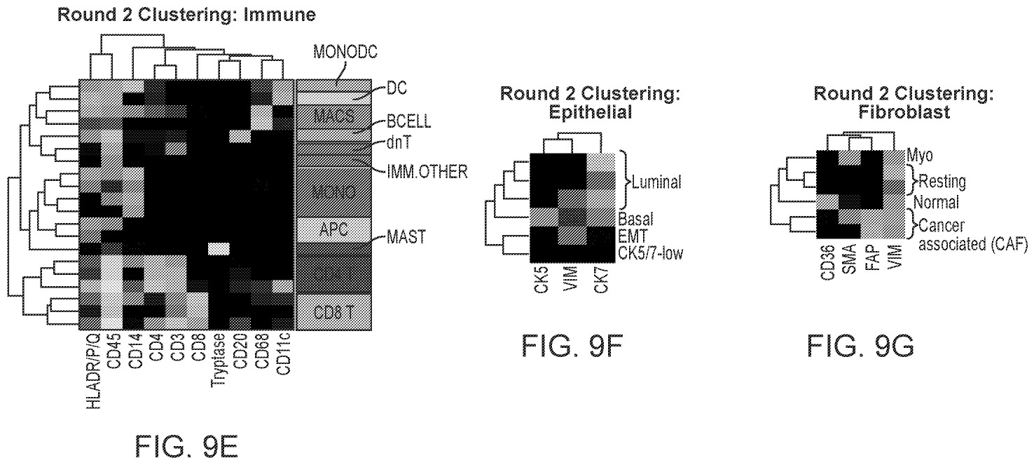

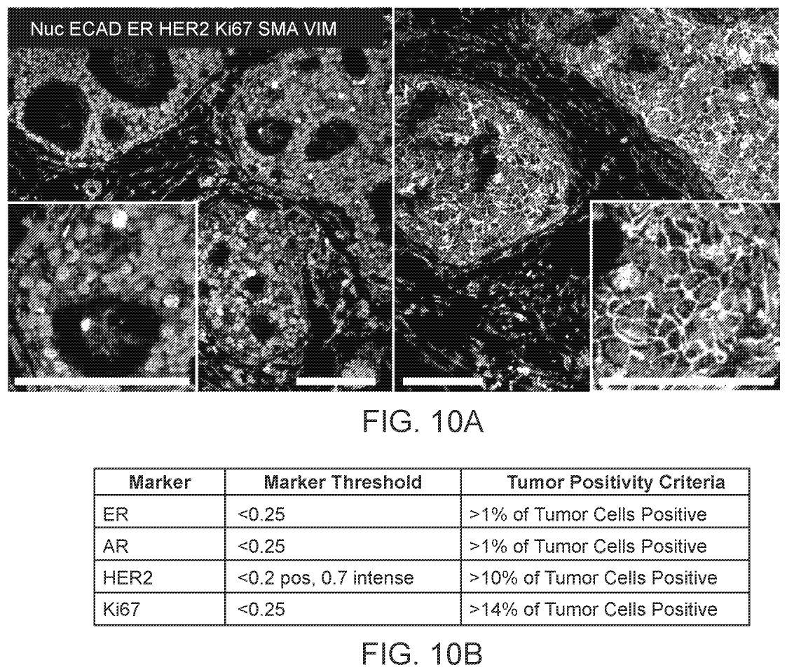

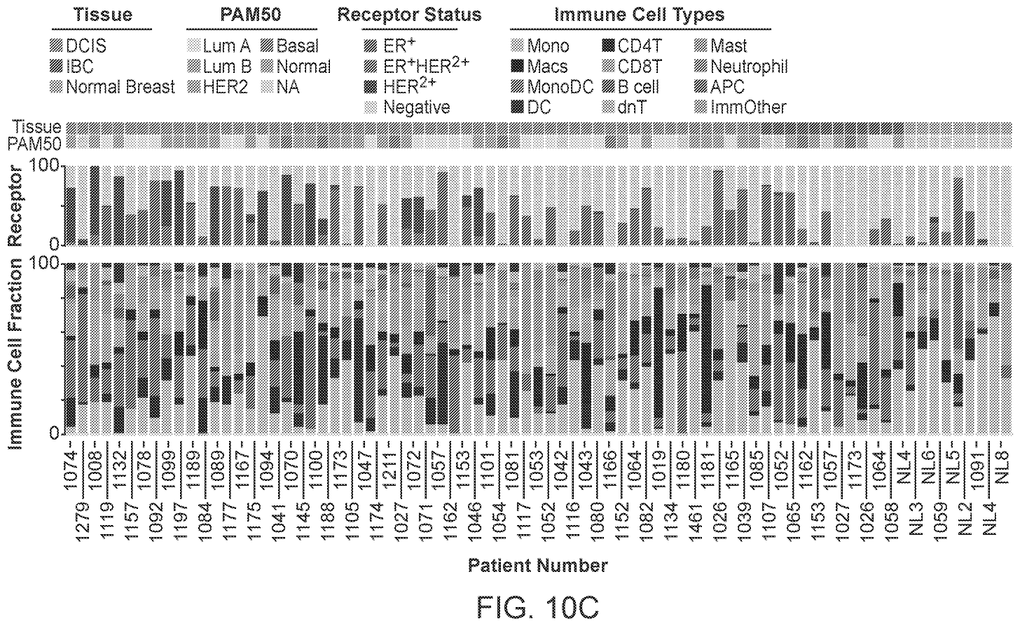

MIBI-TOF imaging was performed on each RAHBT TMA using a 37-plex metal-conjugated antibody staining panel (FIG. 2B, FIG. 7), acquiring one 500×500 μm region of interest per core. A deep learning pipeline (Mesmer) was subsequently used to annotate single cells in each image (mean=875 cells per image, standard deviation=316 cells, FIG. 8, STAR Methods Low-level Image Processing and Single Cell Segmentation). We then used FlowSOM to identify tumor cells, fibroblasts, myoepithelium, endothelium, and 12 types of immune cells (FIG. 2C, FIG. 9A-E). Overall, we assigned 95% of segmented cells (N=69, 151 single cells) to one of these 16 cell classes that had an aggregate frequency range of 0.7-58.3%. To examine how cell type and function varied with respect to tissue structure (FIG. 2D), these data were combined to generate cell phenotype maps (FIG. 2E) and tissue compartment masks (FIG. 2F) demarcating the epithelium, stroma, and myoepithelium.

DCIS epithelial and stromal tissue compartments were predominantly composed of epithelial cells and fibroblasts, respectively, which were each comprised of four major phenotypic subsets. Epithelial cells consisted of luminal (56.9%±33.7), basal (4.4%±6.6), epithelial-to-mesenchymal (EMT, 2.3%±2.8), and CK5/7-low (36.2%±33.5) subsets defined by variable expression of vimentin, CK7, and CK5 (FIG. 2G, H). Fibroblasts consisted of normal fibroblasts (12.1%±15), myofibroblasts (23.5%±16), resting fibroblasts (47%±20.3), and cancer-associated fibroblasts (CAFs; 17.4%±18.2 of fibroblasts) that were defined by variable coexpression of CD36, fibroblast activation protein (FAP), and smooth muscle actin (SMA) (FIG. 2I, J, FIG. 9G, H). Per-patient interrogation of epithelial, fibroblast, and immune cell subsets across DCIS, IBC, and normal breast revealed that all phenotypic subsets were observed in all tissue types, including ER-, HER2-, and AR-defined functional subsets, with primary DCIS tumors showing high interpatient heterogeneity in cellular and PAM50 subtype makeup (FIG. 2K, FIG. S4A-C). These data indicate that beyond the presence of myoepithelial cells, DCIS tumors have a diverse epithelial, stromal, and immune makeup that cannot be differentiated from IBC solely based on the presence of discrete cell types.

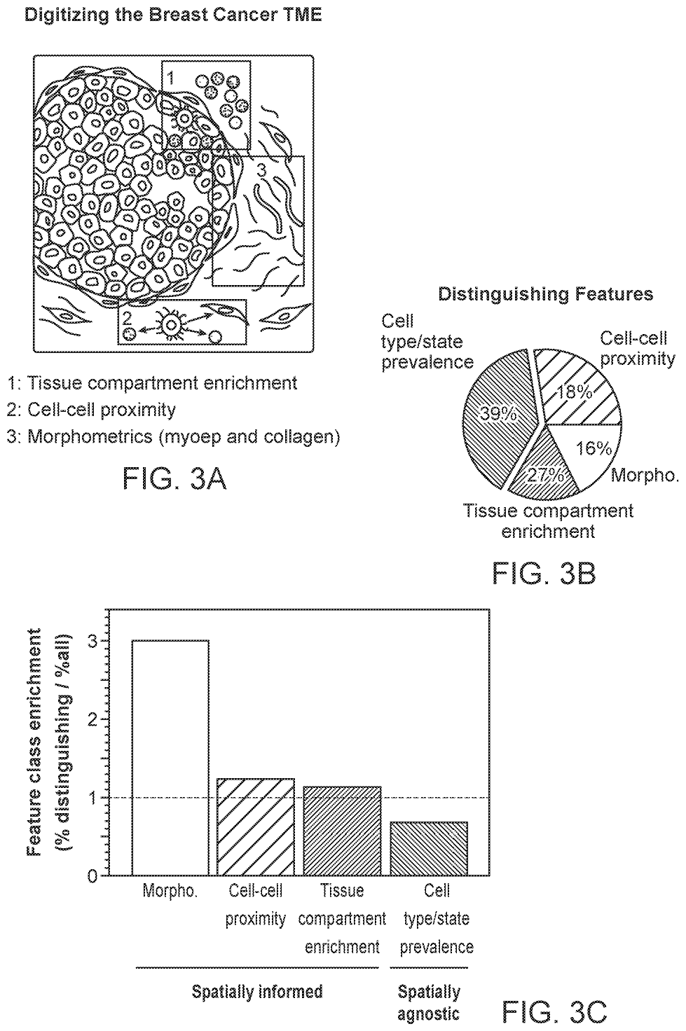

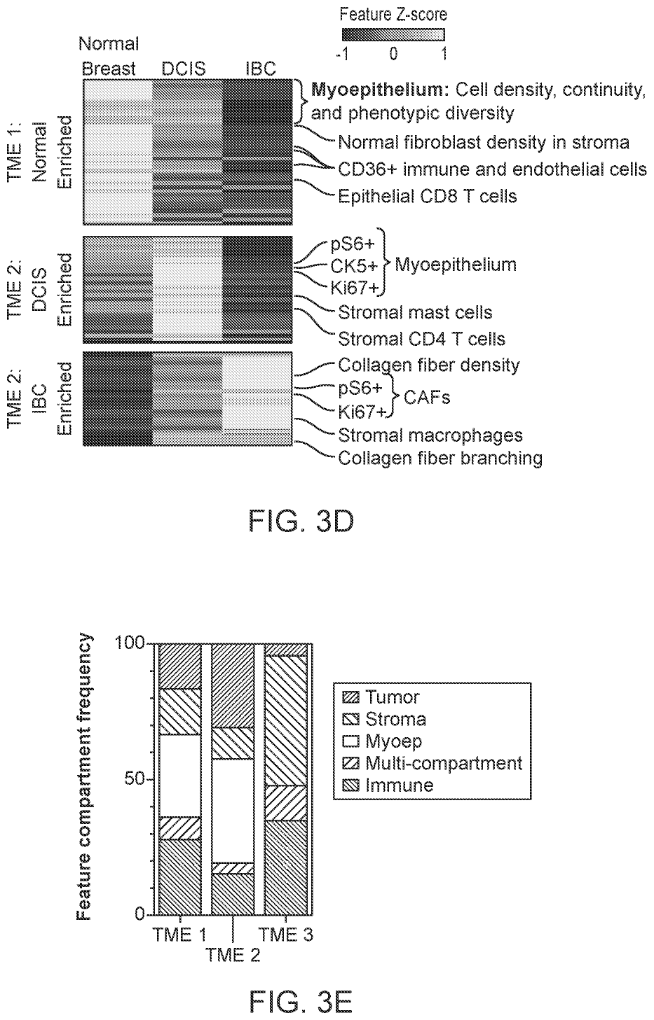

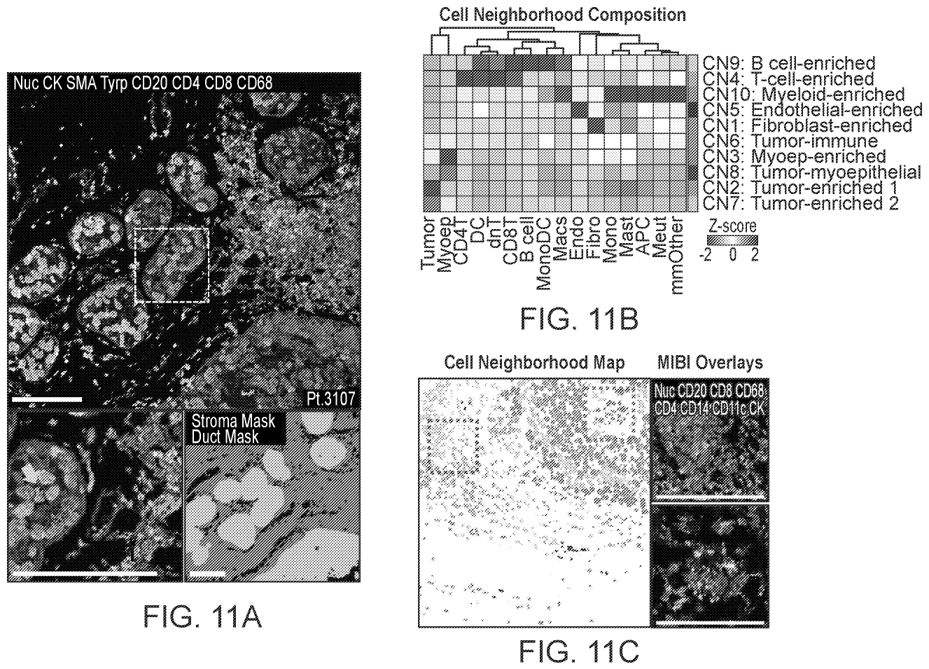

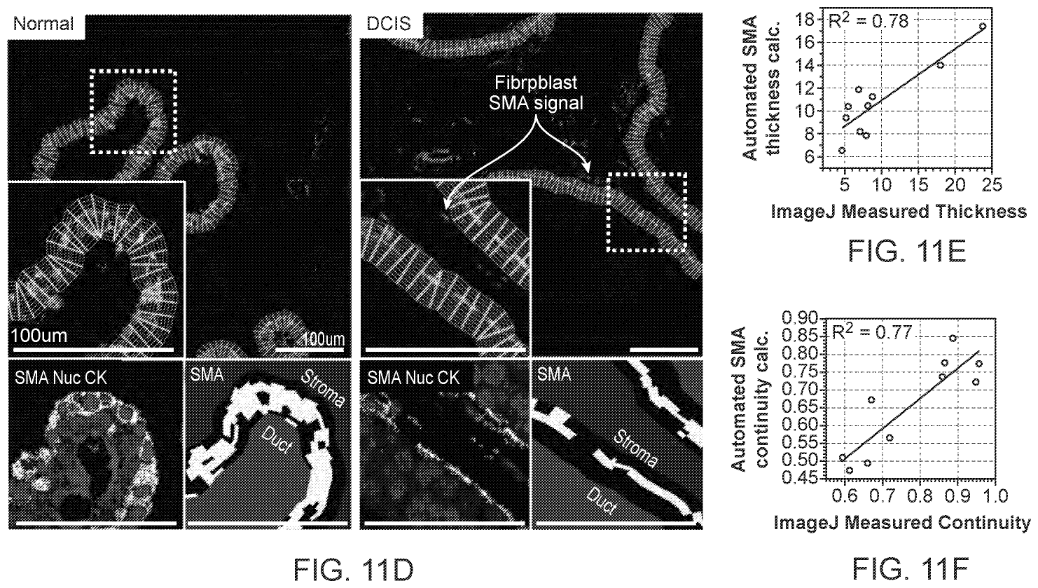

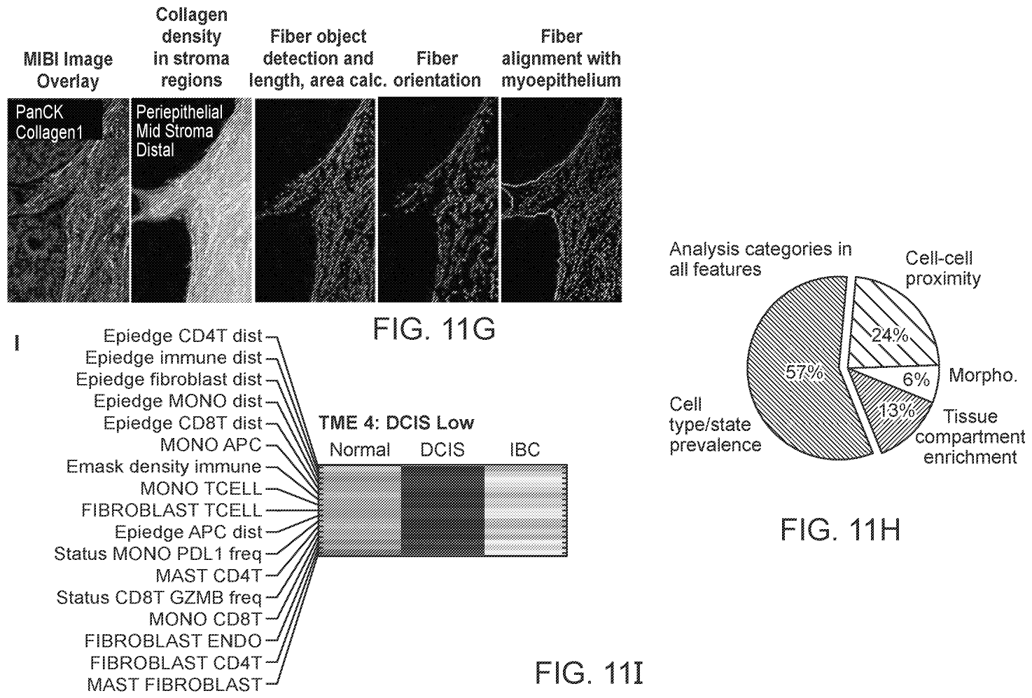

Transition to DCIS and IBC is marked by coordinated changes in the TME. In the previous section, we defined normal, DCIS, and IBC samples in terms of bulk cellular composition in a manner that was agnostic to the spatial location of each cell population. Next, to interrogate potential spatial differentiators of disease state, and to understand how tissue composition, cellular organization, and structure are interrelated, we augmented these compositional data with a description of the spatial distribution of each cell subset within the TME. First, to determine the proportion of each cell population residing within ductal or stromal regions, we used regional masks demarcating the epithelium and stroma to quantify the frequency of each cell type in these regions (Tissue Compartment Enrichment, FIG. 3A, FIG. 11A, STAR Methods Compartment Analysis; Note: due to loss of myoepithelium in IBC, this compartment was not analyzed in these samples). Next, we used two cell-cell proximity metrics—pairwise cell distances and cell neighborhoods—to capture preferential spatial interactions between discrete cell types (Cell-cell proximity, FIG. 3A, FIG. 11B, Start Methods Region Masking).

In addition to this more general cell-centric approach, we also developed custom tools for capturing specific morphologic and phenotypic attributes of the thin monolayer of myoepithelium-encapsulating ductal epithelial cells and the structure of stromal collagen (TME morphometrics, FIG. 3A, FIG. 11D-G, STAR Methods Myoepithelial Continuity and Thickness Analysis, Myoepithelial Pixel Clustering Analysis, Collagen Morphometrics). Taken together, this analysis yielded a digitized TME profile consisting of 433 parameters quantifying both the cellular composition and spatial structure of each patient sample.

We then compared these profiles for normal, DCIS, and IBC tissues to address our first question: how do the composition and structure of the TME change with progression to IBC? We applied the Kruskal-Wallis H test to discern which aspects of tissue composition and structure were significantly distinctive of each clinical group (p<0.05, STAR Methods Distinguishing Feature Analysis). This analysis identified 137 parameters that were preferentially enriched or depleted in normal, DCIS, or IBC tissue, with spatially agnostic (cell type, cell state) and spatially informed metrics accounting for 39% and 61% of differentially expressed parameters, respectively (FIG. 3B, FIG. 11H). Notably, all three categories of spatially informed parameters were overrepresented. For example, morphometrics were three-fold enriched, accounting for 16% of distinguishing parameters but only 5% of all parameters (FIG. 3C).

To organize distinguishing features into interpretable TME signatures, we performed k-means clustering to yield four clusters defining the breast tissue states: TME1, TM E2, and TME3 uniquely distinguished normal, DCIS, and IBC samples, respectively, and TME4 consisted of features that were specifically depleted in DCIS samples (FIG. 11I). Not surprisingly given its enrichment in normal breast, TME1 was typified by myoepithelium with high cellularity, thickness, and continuity (FIG. 3D). Additionally, this robust myoepithelial layer in TME1 was paired with elevated CD36 expression in endothelium and immune cells (FIG. 3D, TME1 “CD36+immune and endothelial cells”), consistent with normative lipid metabolism in homeostatic breast tissue. TME2 was specifically enriched in DCIS tumors and was typified by increased myoepithelial proliferation (% Ki67+), stromal mast cells, and CD4 T cells. Notably, TME2 contained the highest proportion of tumor and myoepithelial parameters (FIG. 3D, TME3 “pS6+, CK5+, Ki67+myoepithelium”), suggesting that the transition to in situ disease involves a coordinated shift in the function of these two lineages (FIG. 3E). IBC-enriched TME3 was stroma-predominant (50%) and had surprisingly few distinctive tumor parameters (4%; FIG. 3E).

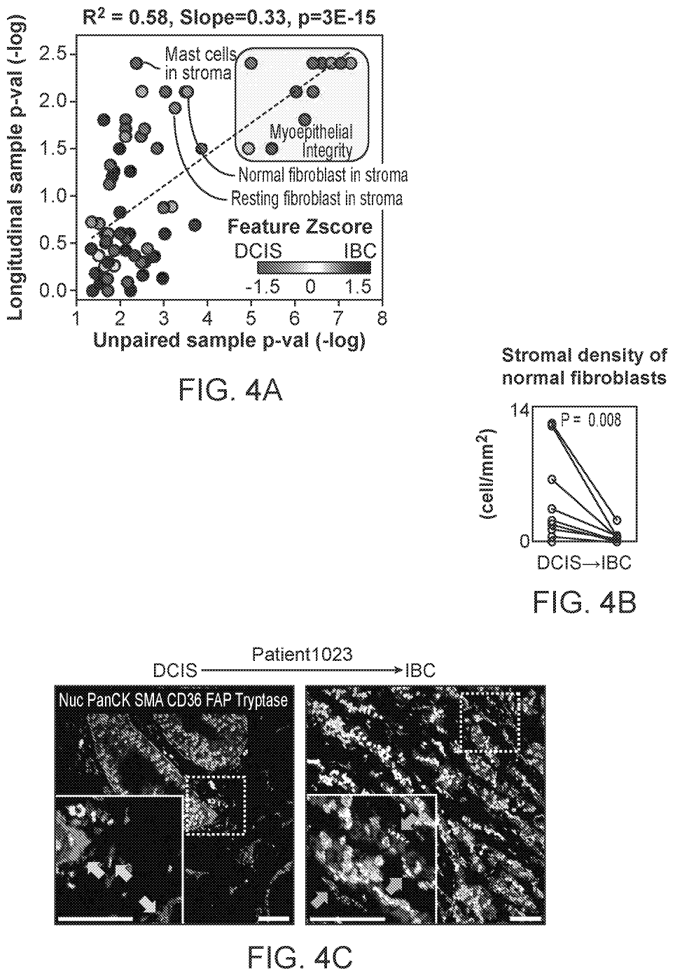

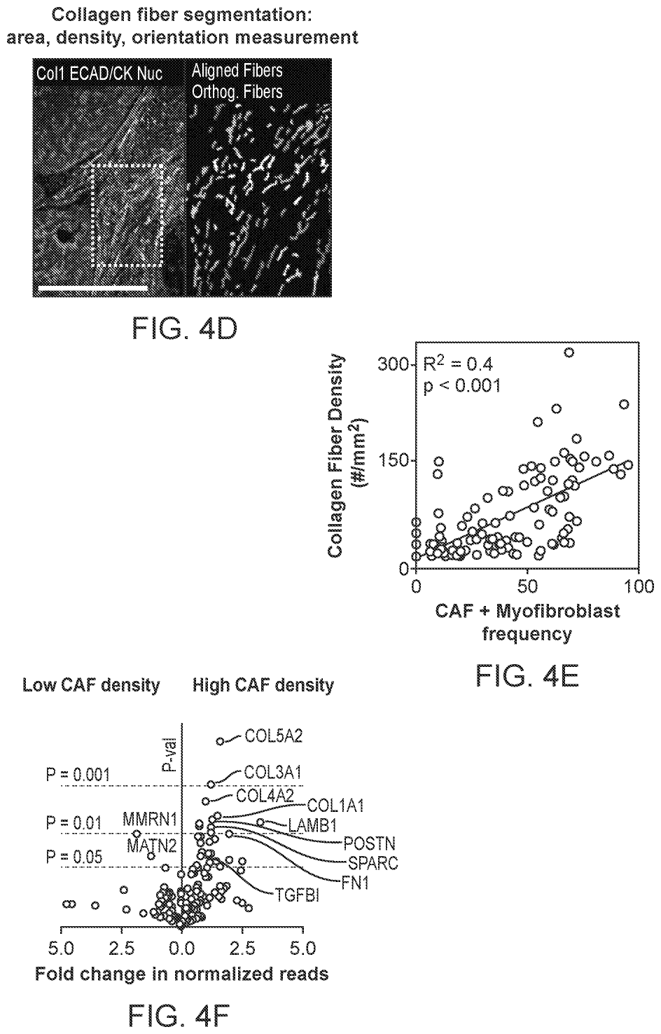

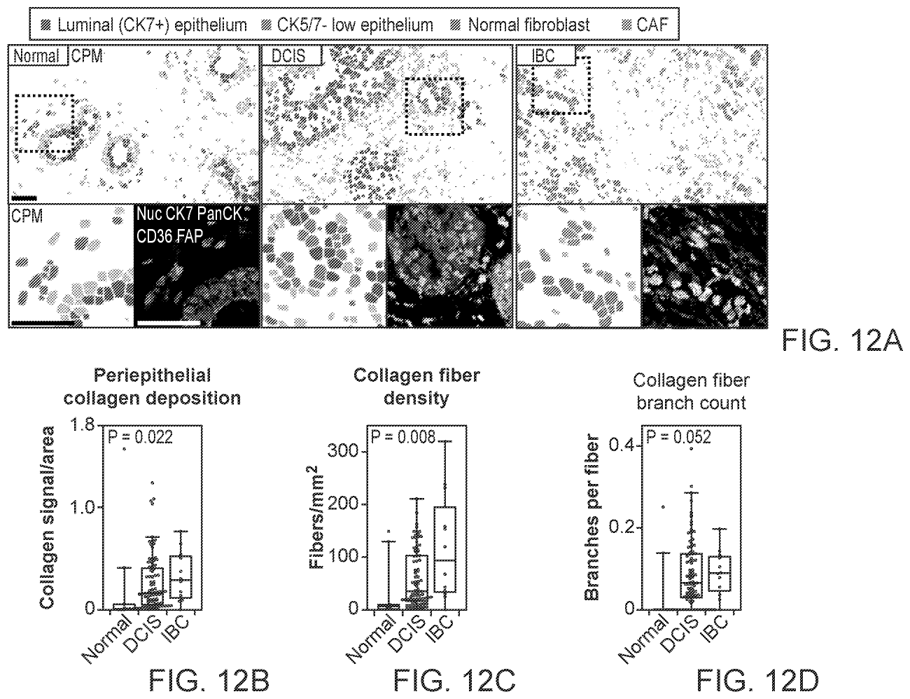

Along these lines, we noted when comparing TME2 and TME3 that—aside from the pathognomonic loss of ductal myoepithelium—the most distinctive property delineating DCIS from IBC samples was an increase in stromal desmoplasia (collagen deposition, CAF frequency, and proliferation). To further evaluate whether these trends reflected changes specific to the interval between a new DCIS diagnosis and ipsilateral invasive relapse, we compared these parameters in a subset of sample pairs in which both DCIS and IBC tissue had been procured longitudinally from the same patient (N=9). We found that the degree of statistical significance in this lesser-powered pairwise analysis and the larger unpaired analysis were linearly correlated (R2=0.58, p=3E-15) and that the salient trends reflected in TME2 and TME3 occurred at the patient level (FIG. 4A). These significant longitudinal changes included a reduction in mast cells, resting fibroblasts, and normal fibroblasts in the stroma between paired patient samples (FIG. 4B), reflecting a transition where normal fibroblasts in primary DCIS samples (FIG. 4C, green arrows) were supplanted by CAFs (FIG. 4C, pink arrows) in patients' subsequent later invasive breast events (FIG. 12A).

To quantify how this shift in fibroblast phenotype relates to the extent of stromal desmoplasia, we compared the shape, length, and density of individual collagen fibers with CAF location, frequency, and phenotype (FIG. 4D, STAR Methods Collagen Morphometrics). Collagen fiber density was linearly correlated with the presence of stromal CAFs and myofibroblasts (R2=0.4, FIG. 4E), suggesting a direct relationship between CAF activation and the extent of collagen fibrillization. Finally, to identify changes in the proportion of collagen isoforms accompanying CAF activation, we compared transcript levels in stroma of CAF high- and low-density tumors using LCM RNAseq. The majority of collagen species were upregulated in CAF-high tumors with COL5A2, COL3A1, and COL1A1 (p<0.01, FIG. 4F). In addition, CAF-high tumors showed increased deposition of fibronectin (FN1; p<0.05), SPARC (p<0.01), and periostin (POSTN; p<0.01), which have been shown to promote a pro-invasive stromal niche.

Identifying DCIS features correlated with risk of invasive progression. We next leveraged both spatially informed and agnostic parameters to examine our second central question: what distinguishes DCIS lesions that later progress to IBC from those that do not? We compared tissue procured at the time of diagnosis in two sets of patients with primary DCIS. The first set, referred to as “progressor”, consisted of 14 patients who had a subsequent ipsilateral invasive recurrence following a diagnosis of pure DCIS (median time to recurrence=9.1 years). The second set, referred to as “non-progressor”, consisted of 44 patients with pure DCIS that did not have a breast event following tumor resection (median time of follow=11.4 years).

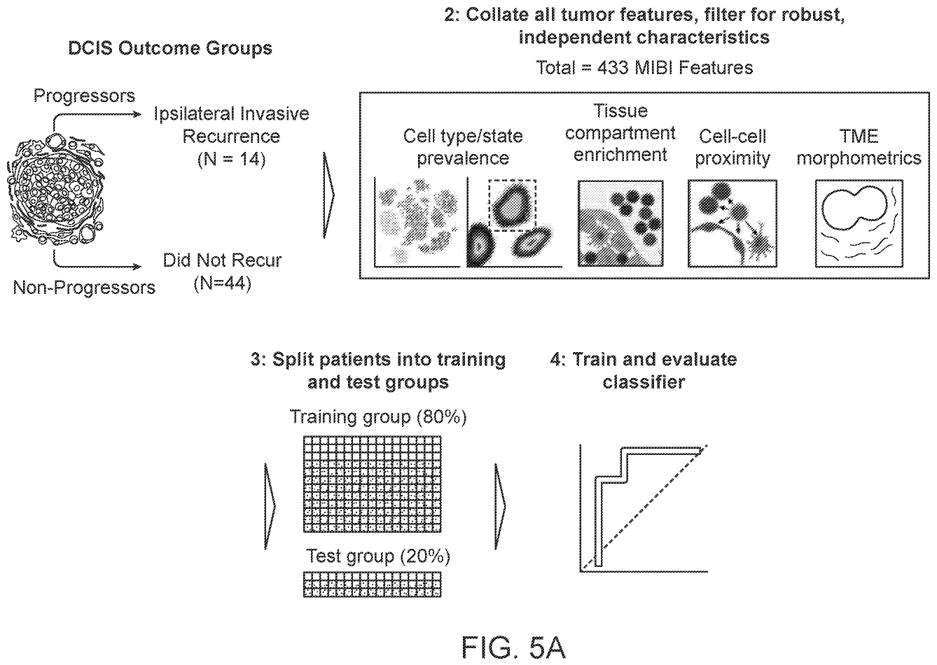

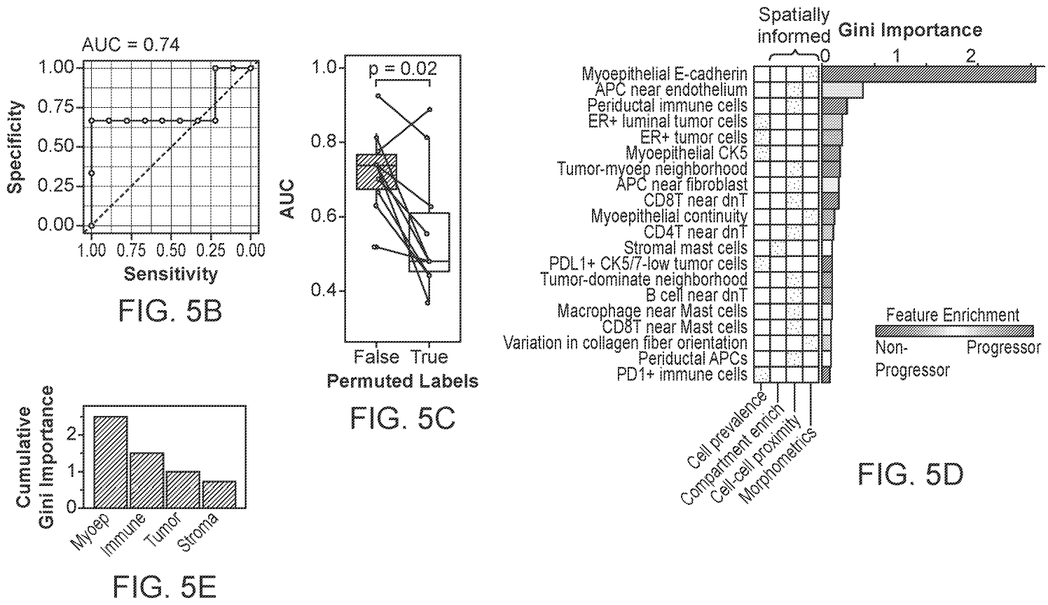

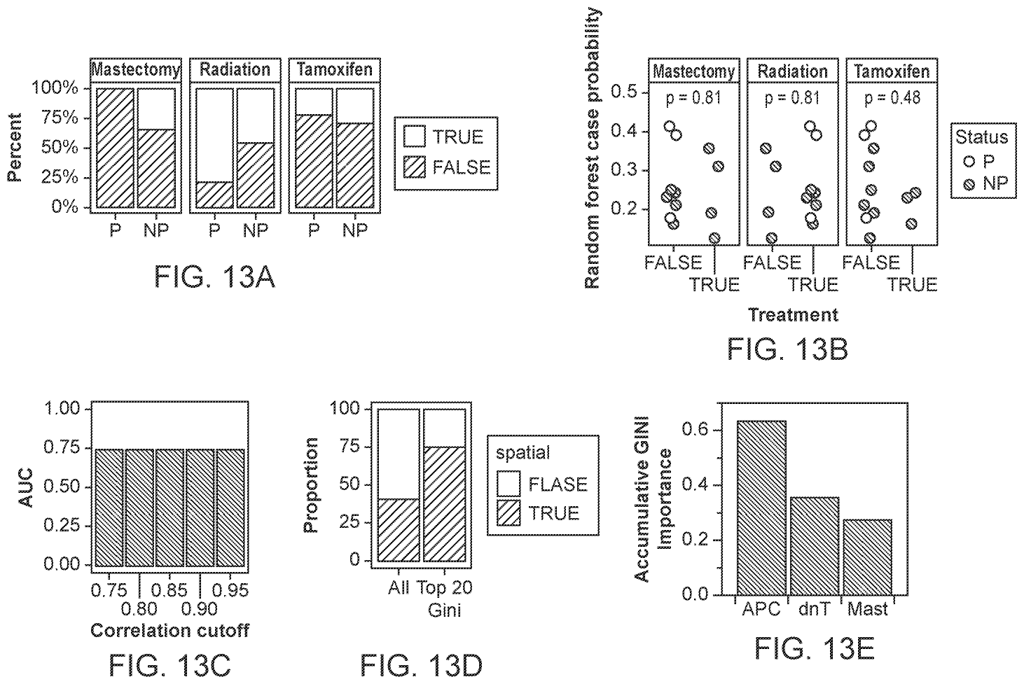

To identify predictive features of the TME, we trained a random forest classifier to predict which patients would relapse with invasive disease based on cell-type prevalence, tissue compartment enrichment, cell-cell proximity, and morphometrics for each sample (FIG. 5A). Although sample size precluded us from being able to eliminate patient demographics and differences in clinical therapy as confounders in this analysis, treatment regimens known to affect recurrence rates (mastectomy, radiation, tamoxifen) were well distributed between the progressor and non-progressor patients (FIG. 13A). Likewise, no significant differences in classifier predictions were identified with respect to these variables (FIG. 13B).

After removing sparse and overly correlated parameters, we randomly split the patient population 80/20 into training and test sets, respectively (FIG. 13C). We evaluated classifier accuracy in the withheld test set, where the model achieved an area under the curve (AUC) of 0.74 (FIG. 5B). To control for variation due to the random partitioning of training and test sets, we repeated this approach with 10 different seeds, resulting in 10 different training test partitions, and maintained a median AUC of 0.74 (FIG. 5C). For additional rigor, we trained classifiers on randomly permuted patient group labels for each seed and compared the distribution of resultant AUCs to the unpermuted models. Pairwise comparison of these replicates demonstrated significantly superior accuracy when using unpermuted data (median AUC of 0.74 (red) vs. 0.48 (blue), p=0.02), demonstrating that the model's predictive power is predicated on the distinct biological features of progressors and non-progressors.

To understand the biology being leveraged by the model to accurately discriminate pre- invasive from indolent DCIS tumors, we ranked the top 20 features based on Gini importance. These features primarily consisted of metrics related to the phenotype of myoepithelium and the spatial distribution of multiple immune cell subsets (FIG. 5D, E). Notably, spatially informed metrics describing cell densities, cell neighborhoods, pairwise cell distances, collagen structure, and multiplexed subcellular features were overrepresented and accounted for 15 of the top 20 metrics in the invasive model (FIG. 7D), while representing less than half of total measured features (FIG. 13D, E).

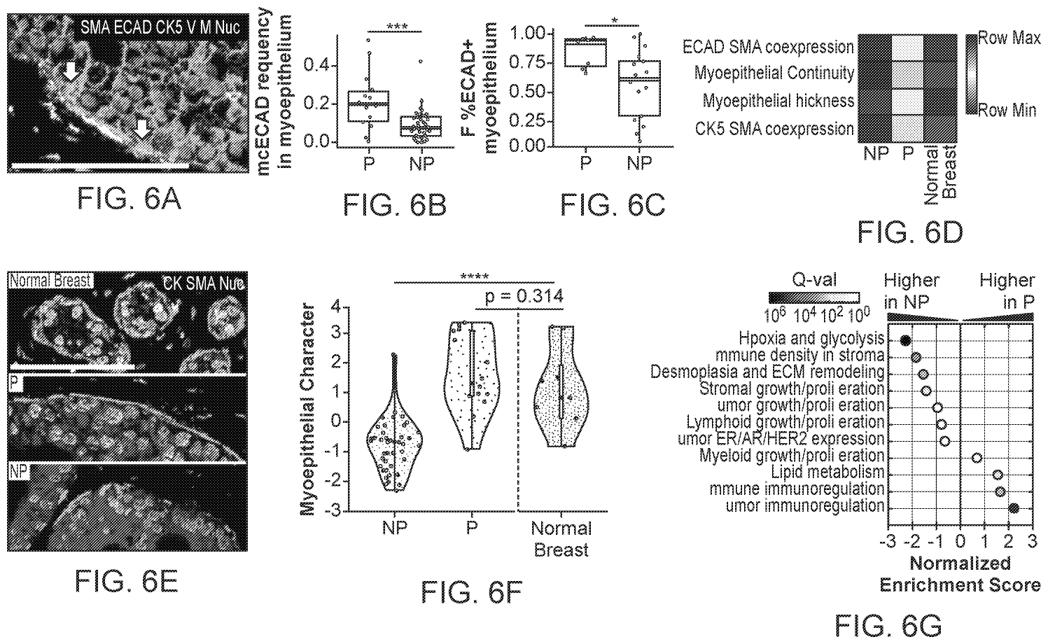

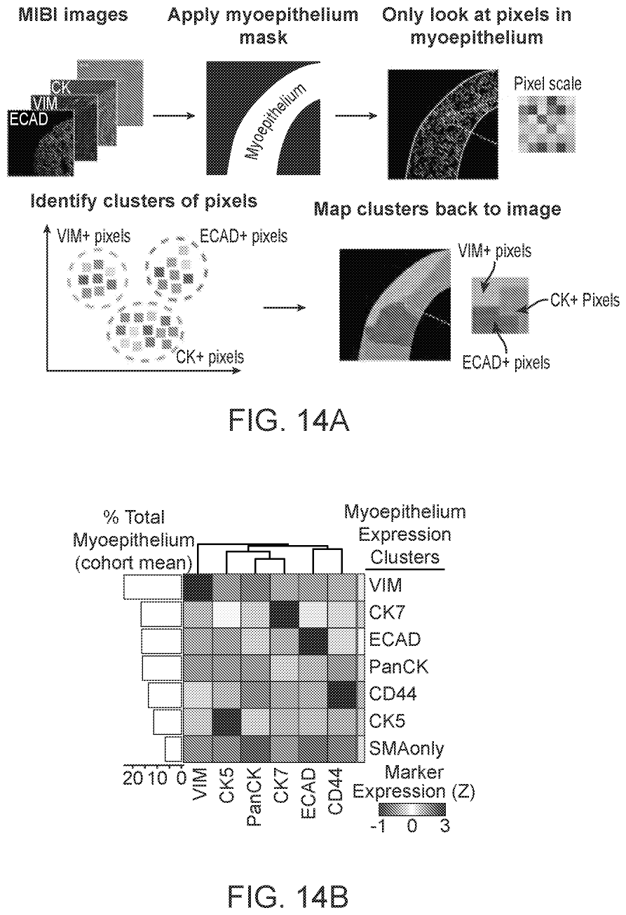

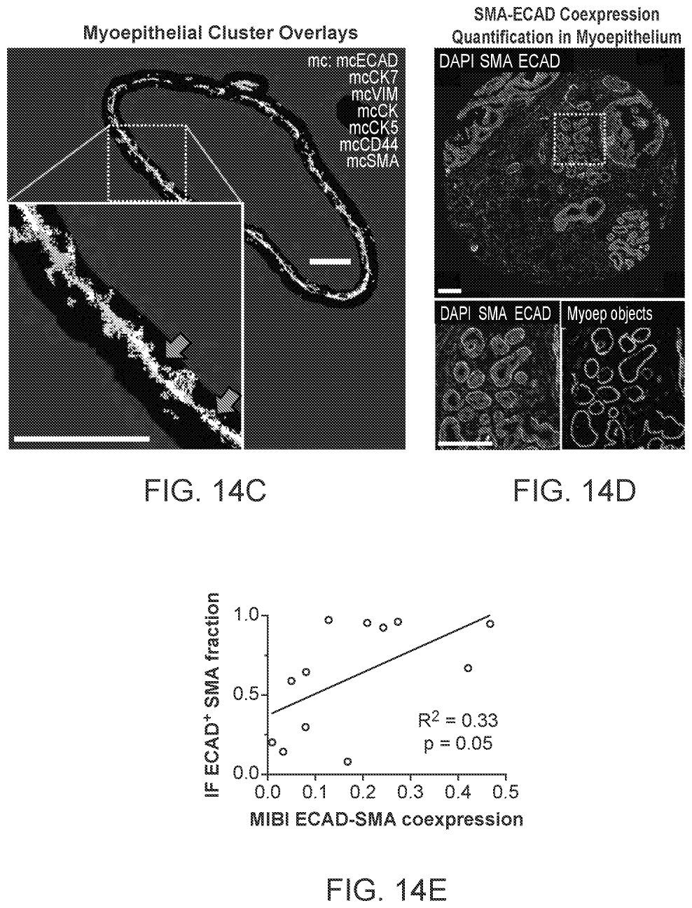

Myoepithelial breakdown and phenotypic change between progressors and non-progressors. In the above analysis, myoepithelial structure and phenotype were overrepresented among the top Gini-ranked classifier features (FIG. 5D), with myoepithelial expression of E-cadherin (ECAD) being the most discriminative feature. This parameter quantifies ECAD co-expression at the pixel level exclusively in periductal SMA-positive pixels (FIG. 6A, pink arrows) and was significantly elevated in progressor samples (p=0.001, FIG. 6B, FIG. 14A-C). We validated this finding using multi-color immunofluorescence for ECAD and SMA. Pixel-level coexpression in immunofluorescence measurements was higher in progressors than non-progressors (p=0.034) and was well correlated with patient-matched values attained by MIBI (FIG. 6C, FIG. 14D, E).

In our analyses comparing normal tissue, DCIS, and IBC, we observed the highest myoepithelial ECAD expression in normal breast tissue (FIG. 3). To our surprise, on comparing normal samples with respect to DCIS clinical subgroups, we found that ECAD expression in normal ductal myoepithelium was more similar to progressor samples than non-progressor samples (FIG. 6D). A similar trend was observed with other morphologic and phenotypic properties: progressor DCIS samples more closely resembled normal samples than non-progressor samples. For example, myoepithelium in non-progressors was thinner and less continuous than in progressor and normal samples (FIG. 6D, E). To examine this difference more comprehensively, we trained a linear discriminant analysis model to differentiate progressors and non-progressors using all myoepithelial parameters exclusively, with only DCIS samples in the training set (STAR Methods Myoepithelial Feature LDA). Composite scores (myoepithelial character) for DCIS samples calculated with the resultant model proficiently separated progressors from non-progressors (progressor mean=1.65±1.32, non-progressor mean=−0.75±0.88, FIG. 6F, left). We then used the trained model to quantify the myoepithelial character of normal samples. In line with FIG. 6D, normal breast samples diverged significantly from non-progressor samples (p=2.64E-4) but were statistically indistinguishable from progressor samples (p=0.314).These data suggest that the loss of normal-like features, reflected in myoepithelial character composite scores, serves a protective function in non-progressors in preventing IBC relapse.

To understand how this loss might influence recurrence outcomes, we used a method derived from geneset enrichment analysis to identify ontologies that were correlated with high or low myoepithelial character (STAR Methods Feature Ontology Enrichment Analysis). Low scores typical of non-progressors were enriched for parameter ontologies relating to hypoxia, glycolysis, stromal immune density, and desmoplasia/remodeling of the extracellular matrix (ECM; FIG. 6G). Conversely, high myoepithelial character scores typically seen in progressors were enriched for immunoregulatory marker expression (PDL1, IDO1, COX2, PD1) in tumor and immune cells (FIG. 6G). Taken together, these results suggest that myoepithelial loss serves a protective, tumor-sensing function that favors fibroblast and immune-cell activation in the surrounding stroma.

Here, we report the first spatial atlas of breast cancer progression. The central focus of this study was to central focus is to characterize features in primary DCIS that are associated with risk of invasive relapse, where tumor cells have breached the duct and invaded the surrounding stroma. Previous work examining breast cancer progression has attributed this transition either to tumor-intrinsic factors or to specific features of stromal cells in the surrounding TME. By simultaneously mapping both of these entities in intact human tissue, we sought to treat the DCIS TME as a single ecosystem in which progression to invasive disease depends on an evolving spatial distribution and function of multiple cell types, rather than on any single cell subset.

Meeting this goal required first assembling a large, well-annotated, and diversified pool of human breast cancer tissue: the RAHBT cohort. This effort was motivated in part by the success of similar works investigating invasive disease (METABRIC, TCGA) that have provided deep insights into breast tumor composition and have served as authoritative resources in breast cancer research (Cancer Genome Atlas Network, 2012). The Breast PreCancer Atlas constructed a unique set of archival human surgical resections that captured the full spectrum of breast cancer progression, from normal tissue, to primary DCIS, and onto patient-paired ipsilateral IBC recurrences. Here, assembling all these cases into TMAs has enabled a one-of-a-kind workflow for multiomics analyses in which genomic, transcriptomic, and proteomic techniques are performed not only on the same samples, but on co-registered serial sections of the same local region of tissue.

Here, we analyzed these TMAs using MIBI-TOF and a 37-marker staining panel to map breast cancer progression and to understand why some patients with DCIS relapse with invasive disease while others do not. Our results show that coordinated transformation of ductal myoepithelium and surrounding stroma plays a central role in determining clinical outcome by establishing a tumor-permissive niche that favors local invasion. Relative to normal tissue, the thin myoepithelial layer in DCIS samples was less phenotypically diverse and more proliferative (FIG. 3D). Curiously, these changes were accompanied by an influx of stromal CD4 T cells and mast cells that subsequently declined in IBC. Aside from the canonical loss of myoepithelium, stromal desmoplasia in IBC was the most consistent, distinctive aspect of invasive progression and was marked by higher numbers of proliferating CAFs and densely aligned fibrillar collagen (FIG. 4).

Typified changes in TME structure and function were not only discriminative of DCIS and IBC, but also separated DCIS progressors from non-progressors. Using 433 spatial and compositional parameters drawn exclusively from original primary DCIS samples, we built a random forest classifier model to predict which patients would relapse with an ipsilateral invasive tumor following initial DCIS diagnosis (AUC=0.74, p=0.02). On examining the relative weighting given to each parameter in the model, two compelling and overarching insights emerged. First, spatially informed metrics relating cell function to structure and morphology were significantly over-represented relative to non-spatial metrics. Second, the most influential features were primarily related to myoepithelium and stroma rather than to the tumor cells themselves.

Given its loss in IBC, ductal myoepithelium has long been thought to act as a barrier that deters local invasion by partitioning in situ carcinoma cells away from the surrounding stroma. Initially, we hypothesized that a more intact and robust myoepithelial barrier resembling normal breast tissue would be protective against invasive progression. Surprisingly, however, our data seem to suggest the opposite: DCIS samples with more continuous myoepithelium and high ECAD expression were at higher risk of ipsilateral invasive recurrence following primary DCIS surgical excision. Retention of these normal-like myoepithelial traits correlated with fewer stromal immune cells and CAFs (FIG. 6G). Conversely, the thin, discontinuous, low-ECAD myoepithelium present in non-progressor tumors was correlated with a more reactive desmoplastic stroma with more immune cells, CAFs, and collagen remodeling. Given the relationships uncovered here between myoepithelial integrity and reactive stromal, our observations are consistent with a model in which a compromised myoepithelial barrier promotes stromal sensing of tumor, which provides protection against future invasive relapse.

Taken together, the analyses reported here deliver a comprehensive, multi-compartmental atlas of preinvasive breast cancer that illustrates the full continuum of tissue structure and function starting from a homeostatic state in normal breast through in situ and invasive disease, including matched longitudinal samples. Combining this comprehensive data set with extensive patient follow-up has enabled identification of tumor features that are associated with risk of invasive relapse in DCIS patients and offers a framework for follow-on analysis.

Methods

Patient Cohort. We utilized a retrospective study cohort of patients from the Washington University Resource of Archival Tissue (RAHBT) that contained two outcome groups: non-progressors, which was composed of patients with DCIS who had no new breast event following resection (median follow-up=11.4 years), and progressors, which was composed of patients with DCIS who had a new ipsilateral invasive breast cancer event following primary DCIS resection (median time to new event=9.1 years). For each progressor, we matched two non- progressors who remained free from recurrent lesions, based on age at diagnosis (±5 years) and type of definitive surgery (mastectomy or lumpectomy). For each DCIS diagnosis, we retrieved primary and recurrent tumor slides and blocks for pathology review, secured a whole slide image of each sample, marked for tissue microarray (TMA) cores, and generated TMA blocks with 84 1.5-mm cores, including additional tonsil and normal breast tissue sourced from reduction mammoplasty.

Median age at diagnosis was 54 years, year of diagnosis was 1986 to 2017, and median time to recurrence with was 9.1 years for invasive lesions and 5.3 years for pre-malignant lesions. For women in the cohort with no recurrence, follow-up extended to 132 months, on average. Treatment of initial DCIS ranged from lumpectomy with radiation (approximately half of cases), lumpectomy with no radiation (20%), and mastectomy with no radiation (30%). The RAHBT cohort is composed of African American women (26%) and white women (74%).

Serial sections (5 μm) of each TMA slide were cut onto glass slides for hematoxylin and eosin (H&E) staining, onto laser-capture slides for LCM-RNAseq (SMART-3SEQ), and cut onto gold- and tantalum-sputtered slides for MIBI-TOF imaging. H&E slides were inspected by a breast cancer pathologist to address DCIS purity and to demarcate regions of DCIS to guide MIBI imaging and laser dissection of epithelial and stromal area. The Stanford Hospital cohort lacked paired LCM-RNAseq analysis.

Antibody Preparation. Antibodies were conjugated to isotopic metal reporters as described previously. Following conjugation, antibodies were diluted in Candor PBS Antibody Stabilization solution (Candor Bioscience). Antibodies were either stored at 4° C. or lyophilized in 100 mM D-(+)-Trehalose dehydrate (Sigma Aldrich) with ultrapure distilled H2O for storage at −20° C. Prior to staining, lyophilized antibodies were reconstituted in a buffer of Tris (Thermo Fisher Scientific), sodium azide (Sigma Aldrich), ultrapure water (Thermo Fisher Scientific), and antibody stabilizer (Candor Bioscience) to a concentration of 0.05 mg/mL. Some metal-conjugated antibodies in this study were used as secondary antibodies targeting hapten groups on hapten-conjugated primary antibodies, including the pairs PDL1-Biotin and Anti-Biotin149Sm, and ER-Alexa488 and Anti-Alexa488142Nd.

Tissue Staining. Tissues were sectioned (5 μm thick) from tissue blocks on gold- and tantalum-sputtered microscope slides. Slides were baked at 70° C. overnight followed by deparaffinization and rehydration with sequential washes in xylene (3×), 100% ethanol (2×), 95% ethanol (2×), 80% ethanol (1×), 70% ethanol (1×), and ddH2O with a Leica ST4020 Linear Stainer (Leica Biosystems). Tissues next underwent antigen retrieval by submerging sides in 3-in-1 Target Retrieval Solution (pH 9, DAKO Agilent) and incubating them at 97° C .for 40 min in a Lab Vision PT Module (Thermo Fisher Scientific). After cooling to room temperature, slides were washed in 1×phosphate-buffered saline (PBS) IHC Washer Buffer with Tween 20 (Cell Marque) with 0.1% (w/v) bovine serum albumin (Thermo Fisher).

Next, all tissues underwent two rounds of blocking, the first to block endogenous biotin and avidin with an Avidin/Biotin Blocking Kit (Biolegend). Tissues were then washed with wash buffer and blocked for 1 h at room temperature with 1×TBS IHC Wash Buffer with Tween 20 with 3% (v/v) normal donkey serum (Sigma-Aldrich), 0.1% (v/v) cold fish skin gelatin (Sigma Aldrich), (v/v) Triton X-100, and 0.05% (v/v) sodium azide. The first antibody cocktail was prepared in 1×TBS IHC Wash Buffer with Tween 20 with 3% (v/v) normal donkey serum (Sigma-Aldrich) and filtered through a 0.1-μm centrifugal filter (Millipore) prior to incubation with tissue overnight at 4° C. in a humidity chamber. Following the overnight incubation slides were washed twice for 5 min in wash buffer. On the second day, antibody cocktail was prepared as described above and incubated with the tissues for 1 h at 4° C. in a humidity chamber. Following staining, slides were washed twice for 5 min in wash buffer and fixed in a solution of 2% glutaraldehyde (Electron Microscopy Sciences) in low-barium PBS for 5 min. Slides were sequentially washed in PBS (1×), 0.1 M Tris at pH 8.5 (3×), ddH₂O (2×), and then dehydrated by serially washing in 70% ethanol (1×), 80% ethanol (1×), 95% ethanol (2×), and 100% ethanol (2×). Slides were dried under vacuum prior to imaging.

MIBI-TOF Imaging. Imaging was performed using a MIBI-TOF instrument (IonPath) with a Hyperion ion source. Xe⁺ primary ions were used to sequentially sputter pixels for a given field of view(FOV). The following imaging parameters were used: acquisition setting: 80 kHz; field size: 500 μm², 1024×1024 pixels; dwell time: 5 ms; median gun current on tissue: 1.45 nA Xe⁺; ion dose: 4.23 nAmp h/mm² for 500×500 μm FOVs.

Low-level Image Processing and Single-cell Segmentation. Multiplexed image sets were extracted, slide background-subtracted, denoised, and aggregate-filtered as previously described. Nuclear segmentation was performed using an adapted version of the DeepCell (Mesmer) CNN architecture. A cell nuclei (“Nuc”) channel that combined HH3 and endogenous phosphorous (P) signal was generated for segmentation input as the nuclear channel, and a combination channel of E-cadherin, PanCK, CD45, CD44, and GLUT1 was used as the membrane channel input. To more effectively capture the range of cell shapes and morphologies present in DCIS, we generated two distinct Deepcell segmentation parameter sets for each image that were then combined for optimal cell detection accuracy. The first used a radial expansion of two pixels from the nuclear border to generate a cell object and a stringent threshold for splitting cells (FIG. 8, Stroma Parameters). The second used a radial expansion of three pixel and more lenient threshold for splitting cells (Epithelial Parameters). We combined these masks using a post-processing step that gave preference to the epithelial segmentation mask, overriding stromal mask-detected objects in the same area. Smaller cells identified by the stromal settings and missed in the epithelial settings were combined to the final cell mask.

Single-cell Phenotyping and Composition. Single-cell expression of each marker was measured through total signal counts in each cell object, normalized by object area. Single-cell data were then linearly rescaled by the average cell area across the cohort, and subsequently as in h-transformed with a co-factor of 5. All mass channels were scaled to 99.9th percentile. In order to assign each cell to a lineage and subsequent cell type, the FlowSOM clustering algorithm was used in iterative rounds with the Bioconductor “FlowSOM” package in R (v.1.16.0). The first clustering round separated cells into 100 clusters (xdim=10, ydim=10), which were assigned to one of five major cell lineages based on well-established combinations of lineage marker expression, including: epithelial cells (PanCK+, ECAD+, CD45−, CK7+/−, VIM+/−), myoepithelial cells (SMA+, CD45−, PanCK+/−, ECAD+/−, CKS+/-, VIM+/−), fibroblasts (VIM+, PanCK−, ECAD−, CK7-, CD45-, SMA+/-, FAP+/-, CD36+/−), endothelial cells (CD31+, VIM+, PanCK−, ECAD−, CK7−, CD45−, SMA+/−), and immune cells (CD45+, PanCK−, ECAD−). Accurate lineage assignment was assessed by reviewing cells from each FlowSOM cluster in image overlays of lineage-defining markers. In clusters with rare, non-canonical combinations of marker expression, cluster assignments were extensively reviewed across images of various tissue types with pathologist assistance, utilizing morphometric and histological organization features in addition lineage marker expression to accurately phenotype the cells. See FIG. 9D for examples of cell reassignment.

Following lineage assignment, each lineage was subclustered to identify immune cell types including B cells (CD20+, CD4+/−), CD4 T cells (CD4T; CD3+, CD4+, CD8−/low), CD8 T cells (CD8T; CD3+, CD8+, CD4−/low), monocytes (Mono; CD14+, CD11c−, CD68−, CD3−), monocyte-derived dendritic cells (MonoDCs; CD14+, CD11c+, HLADR+, CD68−, CD3−), dendritic cells (DCs; CD11c+, HLADR+, CD3−), macrophages (Macs; CD68+, HLADR+, CD14+/−), mast cells (Mast; Tryptase+), double-negative T cells (dnT; CD3+, CD4−, CD8−), and HLADR+APC cells (APC; HLADR+, CD45+/low). CD45+-only immune cells were annotated as “immune other”. Neutrophils were rare in the dataset; they were assigned last based on the positivity threshold (>0.25) of MPO expression in immune cells. Tumor and fibroblast cells were similarly subclustered to reveal phenotypic subsets, including luminal (ECAD+, PanCK+, CK7+), basal (ECAD+, PanCK+, CK5+), epithelial-to-mesenchymal (EMT; ECAD+/−, PanCK+, VIM+), CK5/7-low (ECAD+, PanCK+) tumor cells, and normal (VIM+, CD36+), myo- (VIM+, SMA+), resting (VIM+only), and CAF (VIM+, FAP+) fibroblasts (FIG. 9). Overall, we assigned 94% (N=127,451 of 134,631) of cells to 16 subsets, with the remaining nucleated cells with absent or very low levels of lineage markers assigned as “other”.

Throughout this work cellular data are presented as 1) the frequency of a cell type of its parental lineage across the entire image (e.g., luminal tumor cells as % of total tumor cells in image), 2) a cell type's density within a particular compartment of the image (e.g., 50 fibroblasts per mm2 of stroma (see Region Masking for compartment definition)), or 3) for immune cells, the frequency of immune cell types (of total immune) calculated for both epithelial and stromal regions (e.g. % macrophages of total epithelial immune). To calculate myoepithelial cell density, the number of cells phenotyped as myoepithelium in each image is normalized by the area of the myoepithelial mask in that image.

Region Masking. Region masks were generated to define histologic regions of each FOV including the epithelium, stroma, myoepithelial (periductal) zone, and duct. We removed gold-positive areas, which marked regions of bare slide from holes in the tissue, providing an accurate measurement of tissue area. This area measurement was used to calculate cellular density in specific histologic regions (e.g., fibroblast density in the stroma) to normalize observed cell abundances by the amount of tissue sampled. The epithelial mask was first generated though merging the ECAD and PanCK signals and applying smoothing (Gaussian blur, radius 2 px) and radial expansion (20 px) to incorporate the myoepithelial zone; the insides of ducts were filled. The stromal mask included all of the image area outside of the epithelial mask. Duct masks were generated through the erosion of the epithelial masks by 25 px. The myoepithelial mask was generated by subtracting the duct mask from the epithelial mask, leaving a ˜15 μm-wide periductal ribbon following the duct edge. To calculate the area in each mask, a bare slide mask was generated from the gold (Au) channel and this area was removed from the measurement, and pixel area was converted to mm2 of tissue.

Cellular Spatial Enrichment Analyses. A spatial enrichment approach was used as previously described for enrichment or exclusion across all cell-type pairs. HH3 was excluded from the analysis. For each cell type pair of cell type X and cell type Y, the number of times the centroid of cell X was within a ˜50 μm radius of cell Y was counted. A null distribution was produced by performing 100 bootstrap permutations in which the locations of cell Y were randomized. A z-score was calculated comparing the number of true co-occurrences of cell X and cell Y relative to the null distribution. Importantly, symmetry was assumed: the values of the spatial enrichment of cell X close to cell Y are the same as the values with cell Y close to cell X. For each pair of cell types, the average z-score was calculated across all DCIS FOVs. To analyze cellular associations with the edge of the epithelium, the distances between all cell centroids to the nearest perimeter location of the epithelial mask (described above) were calculated. Cell neighborhoods were produced by first generating a cell neighbor matrix in which each row represents an index cell and columns indicate the relative frequency of each cell phenotype within a 36-μm radius of the index cell. Next, the neighbor matrix was clustered to 10 clusters using k-means clustering, with the number of clusters being determined as the number that best separated distinct immune cell mixtures and tumor/myoepithelial spatial relationships. The neighborhood cellular profile was determined by assessing the mean prevalence of each cell phenotype within a 36-μm radius of the index cell.

Distinguishing Feature Analysis. To determine features that distinguish among normal breast tissue, DCIS, and IBC, means of all 433 features were compared between groups using the Kruskal-Wallis H test. Features with significance under p=0.05 were subsequently clustered using k-means clustering into the 4 TME clusters. For paired analyses, feature means were compared between DCIS and IBC samples from the same patient.

ECM Gene Analysis. To analyze ECM components by gene expression, an ECM gene signature (GO ECM structural constituent, GO:0030021) was downloaded from the GSEA website and used to compare MIBI-identified samples with the top and bottom quartiles of cancer-associated fibroblast density in the stroma. Stromal LCM-RNAseq samples were used for this analysis. Raw reads were normalized with DESeq2 R package (version 1.30.0) (Anders and Huber, 2010) and a paired t-test was compared to the log2 ratio of group means to generate the volcano plot.

Myoepithelial Continuity and Thickness Analysis. To define a window of myoepithelial signal quantitation, we used a topology-preserving operation and defined a curve 5 pixels out from the epithelial mask edge (see Region Masking) and a curve 30 pixels in from the epithelium mask edge; we defined those pixels between these two curves as the myoepithelium mask. We subdivided the outer curve into 5-px arc segments, and for each point on the outer edge between two segments, we found the nearest point on the inner edge, dividing the myoepithelium into a string of quadrilaterals or “wedges”. Wedges were then subdivided along the in-out (of the epithelium) axis into 10 segments. Wedges were merged when both their combined inner and outer edges had an arc length <15 px. We took pre-processed (background subtracted, de-noised) SMA pixels within the mesh and smoothed them with a Gaussian blur of radius of 1. We then calculated the density of SMA signal within each mesh segment as the mean pixel value of smoothed SMA within that mesh segment. This density was then binarized to create a SMA-positivity mesh using a threshold of 0.5 (density>0.5 as positive). The percentage of duct perimeter covered by myoepithelium was calculated by assigning an “SMA-present” variable to each wedge: “0” if no mesh segments in the wedge were positive for SMA, and “1” otherwise. Each wedge was weighted by its area relative to the myoepithelium area. The sum over all wedges of the product of the “SMA-present” variable and the weight was defined as the percent perimeter SMA positivity.

The average (non-zero) thickness of the myoepithelium for each duct was calculated by finding the weighted average “wedge thickness” for SMA-positive wedges (“SMA-present” was 1). The wedge thickness was calculated as the distance between the innermost and outermost positive mesh segments. Positive wedges were weighted by their area relative to the total area of positive wedges. The percent myoepithelial-covered perimeter and average myoepithelial thickness metrics were weighted over meshes (ducts) in a given image by assigning a weight to each duct equal to the total area of the duct myoepithelium divided by the sum of the total areas of all myoepithelium in the image that met a minimum size filter of 7500 px. To assess automated thickness and continuity accuracy, myoepithelial SMA continuity and thickness were quantified manually in 5 progressor and 5 non-progressor SMA images by a board-certified pathologist using ImageJ, blinded to tumor outcome. For continuity, the total periductal perimeter in each image was first quantified by manually outlining each epithelial region. Then, gaps in the myoepithelial layer along this manual outline with no discernable SMA signal where identified. The length for each of these gaps along the periductal perimeter was quantified. Lastly, gap measurements were the summed and divided by total duct perimeter. Smooth muscle thickness was calculated by taking the average of 10 representative linear measurements.

Myoepithelial Pixel Clustering Analysis. Pre-processed (background subtracted, de-noised) images were first subset for pixels within the myoepithelium mask (see Region Masking). Pixels within the myoepithelium mask were then further subset for pixels with SMA expression >0. For all SMA+pixels within the myoepithelium mask, a Gaussian blur was applied using a standard deviation of 1.5 for the Gaussian kernel. Pixels were normalized by their total expression such that the total expression of each pixel was equal to 1. A 99.9% normalization was applied for each marker. Pixels were clustered into 100 clusters using FlowSOM (Van Gassen et al., 2015) based on the expression of six markers: PanCK, CK5, vimentin, ECAD, CD44, and CK7. The average expression of each of the 100-px clusters was found and the z-score for each marker across the 100-px clusters was computed, with a maximum z-score of 3. Using these z-scored expression values, the 100-px clusters were hierarchically clustered using Euclidean distance into six metaclusters. SMA+pixels that were negative for the six markers used for FlowSOM were annotated as the SMA-only metacluster, resulting in a total of seven metaclusters. These metaclusters were mapped back to the original images to generate overlay images colored by pixel metacluster.

Collagen Morphometrics. To identify collagen fibers, background-removed Col1 images were first preprocessed: Col1 pixel intensities were capped at 5, gamma transformed (1 of 2), and contrast enhanced. Images were then blurred via Gaussian with a sigma of 2. While this process enhances fidelity, it yields less clear “0-borders”. This effect was mitigated by generating a “0-region” mask and setting all values to 0 in that region. Then, highly localized contrast enhancement was applied. Since raw fiber signal intensity can vary greatly within a FOV, this step helps enhance locally recognizable—but globally dim—fiber candidates. After this process, contrast was globally enhanced via a reverse gamma transformation (2 of 2). Collagen fiber objects were generated by watershed segmentation on the preprocessed images. An adaptive thresholding method was developed to appreciate variability in total image intensities across the large dataset. A dilated and eroded version of each preprocessed image was produced and subjected to multi-Otsu thresholding. Elevation maps for watershed were generated via the Sobel gradient of a blurred version of the preprocessed images. Once objects were extracted and segmented, length, global orientation, perimeter, and width were computed for each object. Objects that covered low-intensity regions of the image were treated as preprocessing artifacts and were not included in averaging. Average collagen fiber lengths and average collagen branch number were calculated in the entire stromal region. Collagen fiber density (#/area) and total collagen signal were also calculated in specific histological zones defined by distance from the epithelial mask. These zones comprised the periepithelial stroma region (0-20 px from the epithelial edge), mid-stroma region (20-60 px), and distal stroma region (60+px).

Collagen fiber-fiber alignment and fiber-epithelial edge alignment were also measured. For fiber-fiber alignment, fibers were filtered for elongated shape (length>2*width) and alignment was scored as the normalized total paired squared difference over its k nearest neighbors (k=4 was chosen). To accommodate for the elongated shape of these objects, k-nearest neighbors were computed with the ellipsoidal membrane distance, which is the Euclidean centroid distance minus the portion of that distance that lies within the ellipse representation of the object. To compute the myoepithelial-to-fiber (myo-fib) alignment score, the myoepithelial region was identified as the boundary of a manually annotated epithelial mask. This region was then subdivided and labeled as separate objects. The global angle of each object is then compared to the global angle of the K nearest fiber objects, via the same metric described in the fiber-fiber method.

Prediction of Recurrence. To predict recurrence, we compared tissue procured at the time of diagnosis in two sets of patients with primary DCIS. The first set, referred to as “progressor”, consisted of 14 patients who had a new ipsilateral invasive breast event following a diagnosis of pure DCIS (median time to recurrence=9.1 years). The second set, referred to as “non-progressor”, consisted of 44 patients with pure DCIS that did not have a new breast event following primary tumor resection (median time of follow=11.4 years). For each patient, a vector of summary statistics was generated from MIBI data using only images derived from the original lesion. The cohort was split into training (80%) and test (20%) sets; all model optimization and predictor selection steps used only the training set. Any missing values were replaced with the set's predictor mean. Predictors with <12 unique values in the training set were dropped from the analysis. We removed correlated parameters because they could confound predictor importance: all predictors were ranked in importance by performing a Kolmogorov-Smirnov test between progressor and non-progressor within the training set. Greater importance was placed on predictors with lower p-values, with ties broken by weighting predictors with greater effect sizes between patient groups. We quantified pairwise correlation for all predictors (Spearman method). For each group of highly correlated predictors (R>0.85), only the highest-ranked predictor was used in the model. We varied this cutoff and found no difference in model accuracy (FIG. S7E). Two-class random forest probability models (ranger package) (Wright and Ziegler, 2017) were trained to discriminate progressors versus non-progressors. Hyperparameters were tuned on the training set to minimize out-of-bag error. The optimized random forest model was evaluated on the test set and a receiver operating characteristic curve was generated for calculating the area under the curve (pROC package) (Robin et al., 2011) using the model's assigned probability scores. Each predictor's importance was evaluated in the model by its Gini index. All analyses were repeated with 10 distinct random seeds for partitioning patients into training and test sets. For each seed, we additionally trained models using randomly permuted patient group labels (FIG. 5C).

Myoepithelial Immunofluorescence ECAD Quantification. To identify the myoepithelial regions of interest, the SMA channel was first passed through a gaussian filter, and had its maximum intensity capped, to mitigate intense autofluorescent signatures. Next, after being passed through a locally scaled gamma transform to enhance ridge-like features, the channel went through a Meijering ridge filter . To identify candidate myoepithelial “ridges”, the channel was thresholded and all objects were labeled. To filter out distant candidates, their respective distances to a manually annotated mask of the epithelium were measured and gated, only classifying ridges within 80 px as the myoepithelial region. The co-expression of SMA and ECAD was measured in these generated regions.

Myoepithelial Feature Linear Discriminate Analysis (LDA). All myoepithelial features were selected and standardized (mean subtracted and divided by the standard deviation). DCIS (primary and recurring) samples were defined as training data while normal samples were defined as the test set. We then used a dimensionality reduction technique based on LDA on the DCIS-only training set in order to capture the main differences in myoepithelial character between progressors and non-progressors. This supervised method finds the optimal linear combination of a subset of features that maximizes the separation between pre-labeled classes. By combining the myoepithelial features with a progressor/non-progressor label, we separated the DCIS patients in a one-dimensional LDA-generated space (LD1 coordinate) with respect to their progression status. LD1 is therefore the optimized linear combination of the myoepithelial- and SMA-related features for separating progressors from non-progressors. We then calculated LD1 values for our test data—the normal samples based on the trained model. The code for this LDA-based method was provided by (Tsai et al., 2020) and was made available on GitHub. p-values for comparing LD1 distributions between sample types were calculated with the Kruskal-Wallis H test using the Matlab function kruskalwallis.

Feature Ontology Enrichment Analysis. Taking into account DCIS samples only, we calculated the correlation of features with LD1. In this calculation we excluded the 21 features used to define LD1 in the LDA analysis described above. We then sorted the features by correlation with LD1, creating a ranked list of features. Features were also annotated based on belonging to one (or none) of the following functional modules or pathways: Desmoplasia and ECM remodeling (terms: CAFs, MMP9 expression, collagen deposition and fibers), Immune: immunoregulation (immune cells+PD1/PDL1/IDO1/COX2), Lipid metabolism (CD36), Lymphoid: growth/proliferation (CD4T, CD8T, B cell, dnT cell+Ki67/pS6), Myeloid: growth/proliferation (Macs, Mono, MonoDC, DC, APC+Ki67/pS6), Immune density in stroma (immune cell+stroma density), Stroma: growth/proliferation (Fibroblast or endothelium+Ki67/pS6), Tumor: ER/AR/HER2 expression (tumor+ER/AR/HER2), Tumor: immunoregulation (tumor+PDL1/IDO1/COX2), Tumor: growth/proliferation (tumor+Ki67/pS6), and Hypoxia and Glycolysis (HIF1a+GLUT1). This ranked list of features combined with their annotations into pathways was used to perform geneset enrichment analysis (GSEA) using the R package FGSEA. This procedure identified functionally related groups of features that were enriched either among the features highly correlated with LD1 or significantly anti-correlated with LD1.

Statistical Analysis. All statistical analyses were performed using GraphPad Prism (9.1.0), Matlab (2016b), or R (1.2.5033). Grouped data are presented with individual sample points throughout, and where not applicable, data are presented as mean and standard deviation. For determining significance, grouped data were first tested for normality with the D'Agostino & Pearson omnibus normality test. Normally distributed data were compared between two groups with the two-tailed Student's t-test. Non-normal data were compared between two groups using the Mann—Whitney test. Multiple groups were compared using the Kruskal-Wallis H test, with Q-values used for feature selection.

Software. Image processing was conducted with Matlab 2016a and Matlab 2019b. Data visualization and plots were generated in R with ggplot and pheatmap packages, in GraphPad Prism, and in Python using the scikitimage, matplotlib, and seaborn packages. Representative images were processed in Adobe Photoshop CS6. Schematic visualizations were produced with Biorender. R packages used for GSEA were AnnotationDbi (1.52.0) and org.Hs.eg.db, (3.12.0), clusterProfiler (3.19.0), msigdbr (7.2.1), for C2 curated datasets. Python packages used for spatial enrichment analysis and collagen morphometrics were sckikit-image, pandas, numpy, xarray, scipy, stats models.

Data and Code Availability. All custom code used to analyze data is available through our Github repository and all processed images and annotated single-cell data will be made available on a Human Tumor Atlas Network public repository and are present as single marker Tiffs in a public Zenodo repository.

TABLE 1
Feature Corr. with LD1

| | | | p_val_progressor_ |
|---|---|---|---|
| Feature | cor_with_ld1 | Z_cor | vs_nonprogressor |
| Status_immune_PD1_freq | −0.43046544 | 3.727193543 | 0.195016672 |
| Status_TUMOR_Basal_PDL1_freq | −0.396197323 | 3.316088779 | 0.016055961 |
| Status_TUMOR_EMT_HIF1a_freq | 0.367960348 | 2.977337903 | 0.029722989 |
| Status_MONO_HIF1a_freq | 0.353511959 | 2.804004726 | 0.049732605 |
| Status_TUMOR_CK57low_PDL1_freq | −0.326014076 | 2.47412052 | 0.101111744 |
| Status_APC_CD36_freq | −0.322712233 | 2.43450926 | 0.139154925 |
| Emask_density_MACS | −0.315215774 | 2.344576389 | 0.061052328 |
| Status_immune_HIF1a_freq | 0.31416298 | 2.331946323 | 0.169923707 |
| Epiedge_immune_dist | −0.305095858 | 2.223170669 | 0.035047096 |
| Emask_density_TCELL | −0.299459299 | 2.15555049 | 0.342289721 |
| Emask_density_immune | −0.295083577 | 2.103056202 | 0.049640213 |
| Wholeimage_lineagefreq_CAF | 0.294385117 | 2.094676986 | 0.298684486 |
| Status_APC_PDL1_freq | −0.294348352 | 2.094235917 | 0.100252214 |
| Status_TCELL_GLUT1_freq | 0.292870577 | 2.076507475 | 0.107026624 |
| Neighborhood_frequency_clust8 | −0.292257594 | 2.069153693 | 0.040040294 |
| Status_DC_CD36_freq | −0.291417594 | 2.059076462 | 0.609545552 |
| MONO_FIBROBLAST | −0.282235723 | 1.948924185 | 0.233900574 |
| Status_TUMOR_Basal_MMP9_freq | −0.280480543 | 1.927867795 | 0.109930336 |
| Status_endo_CD36_freq | −0.279833585 | 1.920106431 | 0.806041134 |
| Status_TCELL_IDO1_freq | −0.279584691 | 1.917120522 | 0.453384192 |
| Status_MYOFIBRO_MMP9_freq | −0.275096292 | 1.863274481 | 0.001178073 |
| Smask_density_NEUT | 0.271455551 | 1.819597564 | 0.984802876 |
| Smask_lineagefreq_MAST | −0.267244039 | 1.769073261 | 0.423927353 |
| Smask_density_APC | 0.26680184 | 1.763768327 | 0.702750321 |
| Smask_density_TCELL | 0.266682216 | 1.762333229 | 0.823806513 |
| Emask_density_CD8T | −0.264861437 | 1.740489871 | 0.510969883 |
| Status_FIBRO_VIMonly_MMP9_freq | 0.25746396 | 1.651744464 | 0.11558485 |
| Epiedge_fibroblast_dist | −0.253611061 | 1.605522339 | 0.027901547 |
| Smask_density_immune | 0.252513743 | 1.592358128 | 0.689334918 |
| FIBROBLAST_BCELL | −0.251068969 | 1.575025588 | 0.45967824 |
| Status_MACS_HIF1a_freq | 0.250727456 | 1.570928558 | 0.355494358 |
| APC_FIBROBLAST | −0.248177499 | 1.540337451 | 0.403206303 |
| Wholeimage_lineagefreq_TUMOR_Basal | 0.246800476 | 1.523817709 | 0.456131216 |
| Status_NORMFIBRO_Ki67_freq | 0.245399032 | 1.507004988 | 0.089619064 |
| MAST_MONODC | 0.243450347 | 1.483627169 | 0.022037933 |
| Status_endo_Ki67_freq | 0.24272556 | 1.47493211 | 0.355494358 |
| Status_BCELL_pS6_freq | 0.242134899 | 1.467846119 | 0.832445651 |
| Status_immune_GZMB_freq | 0.241366276 | 1.458625166 | 0.359837325 |
| MACS_BCELL | 0.240785061 | 1.451652501 | 0.651375199 |
| Emask_lineagefreq_TCELL | −0.240286201 | 1.445667822 | 0.456682366 |
| Status_FIBRO_VIMonly_IDO1_freq | 0.239721817 | 1.438897063 | 0.089632788 |
| Status_TUMOR_CK57low_HIF1a_freq | 0.239599154 | 1.437425512 | 0.175517454 |
| Smask_density_total_cells | 0.233759945 | 1.367374204 | 0.702767738 |
| Status_MONO_IDO1_freq | 0.231963795 | 1.345826306 | 0.265087551 |
| Status_tumor_pS6_freq | 0.231553389 | 1.34090278 | 0.178741565 |
| Wholeimage_lineagefreq_NORMFIBRO | −0.23121716 | 1.336869138 | 0.629842079 |
| Emask_lineagefreq_NEUT | 0.231114786 | 1.335640988 | 0.623988084 |
| TCELL_CD8T | 0.229085784 | 1.311299631 | 0.037259575 |
| Status_tumor_HIF1a_freq | 0.227970254 | 1.297916941 | 0.154106469 |
| Status_APC_HIF1a_freq | 0.225990588 | 1.274167457 | 0.840034336 |
| Status_CAF_Ki67_freq | 0.225674399 | 1.27037423 | 0.148791816 |
| Status_TUMOR_Lumi0l_pS6_freq | 0.220647533 | 1.210068356 | 0.047593088 |
| MONODC_BCELL | 0.218903829 | 1.189149641 | 0.842086624 |
| Status_BCELL_FAP_freq | 0.217295236 | 1.169851808 | 0.975815997 |
| Smask_lineagefreq_MACS | −0.216725704 | 1.163019297 | 0.071306911 |
| CD8T_BCELL | 0.216027097 | 1.154638311 | 0.772335478 |
| Status_FIBRO_VIMonly_CD44_freq | −0.215379816 | 1.146873065 | 0.77816394 |
| Status_TUMOR_Basal_HIF1a_freq | 0.214886908 | 1.140959791 | 0.246806061 |
| Status_TUMOR_Basal_GLUT1_freq | −0.214567167 | 1.137123954 | 0.165037083 |
| Status_endo_GLUT1_freq | 0.213344214 | 1.122452531 | 0.287236191 |
| Status_MACS_PDL1_freq | −0.213244597 | 1.121257464 | 0.173439544 |
| Neighborhood_frequency_clust7 | −0.212818821 | 1.116149546 | 0.029220636 |
| Status_TUMOR_Lumi0l_HIF1a_freq | 0.20599919 | 1.034336392 | 0.122817778 |
| Status_MACS_GLUT1_freq | −0.203754079 | 1.00740244 | 0.743228122 |
| Smask_lineagefreq_NEUT | 0.203121361 | 0.999811909 | 0.856397888 |
| CD4T_BCELL | 0.203116669 | 0.999755618 | 0.570084477 |
| Status_endo_IDO1_freq | 0.202883537 | 0.996958803 | 0.177826503 |
| Neighborhood_frequency_clust10 | 0.202423179 | 0.991436013 | 0.423984382 |
| MAST_BCELL | −0.199781395 | 0.959743292 | 0.833227594 |
| Wholeimage_lineagefreq_TUMOR_EMT | −0.199631883 | 0.957949636 | 0.560201631 |
| Status_MYOFIBRO_IDO1_freq | −0.199333377 | 0.954368543 | 0.108672382 |
| Status_tumor_COX2_freq | −0.198441224 | 0.943665644 | 0.30547554 |
| TCELL_BCELL | 0.198160747 | 0.94030084 | 0.115559598 |
| Smask_density_CAF | 0.196858631 | 0.924679727 | 0.391482176 |
| Neighborhood_frequency_clust6 | −0.193507271 | 0.884474425 | 0.689334918 |
| APC_ENDO | −0.193429372 | 0.8835399 | 0.027891922 |
| Status_MONO_MMP9_freq | 0.193105113 | 0.879649847 | 0.326863979 |
| Emask_density_NEUT | 0.192917954 | 0.877404561 | 0.544814491 |
| Status_CD8T_HIF1a_freq | 0.191569213 | 0.861224099 | 0.908163795 |
| Status_DC_HIF1a_freq | 0.189149951 | 0.832200914 | 0.594147495 |
| Status_fibroblast_Ki67_freq | 0.188328112 | 0.822341548 | 0.059411675 |
| Neighborhood_frequency_clust3 | −0.18821943 | 0.821037717 | 0.330907377 |
| Smask_density_NORMFIBRO | −0.187634289 | 0.814017947 | 0.682266368 |
| Neighborhood_frequency_clust9 | 0.185424207 | 0.787504229 | 0.34243831 |
| Wholeimage_ratio_CD4CD8_corrected | 0.184470313 | 0.77606063 | 0.985437046 |
| Status_MYOFIBRO_Ki67_freq | 0.18415092 | 0.772228968 | 0.273167536 |
| Status_TUMOR_CK57low_IDO1_freq | −0.184145135 | 0.772159567 | 0.527733282 |
| Status_BCELL_PD1_freq | −0.182392964 | 0.751139273 | 0.917641113 |
| midstroma_thick_avg_object_areas | 0.180832451 | 0.732418245 | 0.14105307 |
| ENDO_DC | −0.178849704 | 0.7086318 | 0.401969715 |
| Emask_density_MONODC | −0.177783029 | 0.695835208 | 0.429094569 |
| MAST_NEUT | −0.177340212 | 0.690522866 | 0.600399062 |
| Smask_density_MACS | −0.174455147 | 0.65591156 | 0.179130567 |
| Status_TCELL_PD1_freq | −0.171635567 | 0.622085869 | 0.360453842 |
| ENDO_CD8T | −0.170901294 | 0.61327701 | 0.248192807 |
| MONO_APC | −0.169953867 | 0.601911003 | 0.963747436 |
| MACS_MONODC | −0.169093891 | 0.591594119 | 0.259128903 |
| Status_TUMOR_Lumi0l_PDL1_freq | −0.168552975 | 0.585104904 | 0.140086488 |
| Status_CD4T_HIF1a_freq | 0.168005159 | 0.578532908 | 0.981593648 |
| Smask_density_CD4T | 0.167862526 | 0.576821786 | 0.447600291 |
| Emask_lineagefreq_DC | −0.166938956 | 0.565741984 | 0.46428454 |
| Status_CD4T_MMP9_freq | −0.166324897 | 0.558375285 | 0.794992009 |
| Status_tumor_PDL1_freq | −0.166323622 | 0.558359994 | 0.075759258 |
| midstroma_fiber_density | 0.16556056 | 0.549205762 | 0.771255575 |
| Smask_density_BCELL | 0.165024426 | 0.542773917 | 0.872302845 |
| midstroma_area_normalized_intensity | 0.164629815 | 0.538039877 | 0.743611492 |
| DC_NEUT | −0.164293787 | 0.534008651 | 0.947905031 |
| Allstroma_avg_branch_count | 0.162412124 | 0.511434879 | 0.674955645 |
| Status_NEUT_HIF1a_freq | 0.161960353 | 0.506015113 | 0.855082539 |
| MACS_NEUT | −0.161694739 | 0.502828619 | 0.666732446 |
| MAST_MACS | 0.161421669 | 0.499552675 | 0.098716788 |
| Status_FIBRO_VIMonly_GLUT1_freq | 0.159441407 | 0.475796032 | 0.168983928 |
| TCELL_CD4T | 0.159214534 | 0.473074305 | 0.104993212 |
| MACS_TCELL | 0.158942817 | 0.469814593 | 0.077917684 |
| Status_TUMOR_Basal_HER2_freq | 0.15816991 | 0.460542256 | 0.07490084 |
| Status_MYOFIBRO_GLUT1_freq | 0.157699697 | 0.454901243 | 0.358074412 |
| Status_TUMOR_CK57low_CD44_freq | −0.156929826 | 0.445665321 | 0.448793673 |
| distalstroma_thick_avg_object_areas | 0.156767045 | 0.44371248 | 0.927608566 |
| Status_TUMOR_EMT_AR_freq | −0.156403382 | 0.439349721 | 0.33129631 |
| Status_fibroblast_HIF1a_freq | 0.155111917 | 0.423856389 | 0.778550933 |
| periepithelial_area_normalized_intensity | 0.153987006 | 0.410361149 | 0.813261561 |
| Status_endo_pS6_freq | −0.153242553 | 0.401430165 | 0.258232244 |
| Status_FIBRO_VIMonly_HLADR_freq | 0.153143124 | 0.400237346 | 0.329360533 |
| Status_TUMOR_EMT_PDL1_freq | −0.15296663 | 0.398119999 | 0.095680583 |
| Status_TUMOR_Lumi0l_HER2intense_freq | 0.152425456 | 0.391627684 | 0.071856108 |
| Status_MONO_PD1_freq | 0.151998426 | 0.386504725 | 0.630324101 |
| APC_BCELL | −0.149671119 | 0.358584696 | 0.739750499 |
| Status_CD8T_GLUT1_freq | 0.149593223 | 0.357650198 | 0.216208614 |
| Status_NEUT_GLUT1_freq | 0.149531467 | 0.356909329 | 0.953148347 |
| FIBROBLAST_NEUT | −0.149176944 | 0.352656219 | 0.569814649 |
| MONO_BCELL | −0.146748895 | 0.323527609 | 0.739750499 |
| APC_TCELL | 0.146601105 | 0.321754616 | 0.985196646 |
| Status_CD8T_MMP9_freq | 0.145746219 | 0.311498795 | 0.879543426 |
| DC_CD8T | −0.145389251 | 0.307216352 | 0.858997712 |
| Status_DC_PDL1_freq | −0.144918001 | 0.301562905 | 0.222363364 |
| MAST_MONO | 0.144811533 | 0.300285637 | 0.073327171 |
| Status_TUMOR_CK57low_COX2_freq | −0.14466203 | 0.2984921 | 0.663043197 |
| Wholeimage_lineagefreq_TUMOR_CK57low | −0.144388135 | 0.295206254 | 0.445359648 |
| CD8T_CD4T | 0.143442106 | 0.283857014 | 0.867378819 |
| Smask_density_MONO | 0.14330871 | 0.282256701 | 0.256019235 |
| Status_CAF_IDO1_freq | 0.143251724 | 0.281573056 | 0.276032454 |
| immune_shannon | 0.143149685 | 0.280348929 | 0.898785538 |
| Smask_density_MONODC | 0.143006702 | 0.2786336 | 0.76716621 |
| Status_BCELL_HIF1a_freq | 0.142088765 | 0.267621371 | 0.951662473 |
| Status_DC_MMP9_freq | −0.14207276 | 0.26742936 | 0.587620431 |
| Emask_lineagefreq_MACS | −0.141402175 | 0.259384547 | 0.182891457 |
| Status_NEUT_MMP9_freq | 0.141067518 | 0.255369769 | 0.917799255 |
| Status_MONODC_HIF1a_freq | 0.140157229 | 0.244449295 | 0.326861614 |
| Status_tumor_HER2intense_freq | 0.139776983 | 0.239887585 | 0.209677671 |
| periepithelial_thick_avg_object_areas | 0.139367713 | 0.234977691 | 0.956510868 |
| Status_tumor_HER2_log2fc_int_vs_pos | 0.138890549 | 0.229253298 | 0.460567265 |
| Smask_lineagefreq_BCELL | 0.138884813 | 0.229184479 | 0.805326997 |
| CD4T_NEUT | −0.13850943 | 0.224681118 | 0.757515448 |
| APC_CD8T | −0.137176394 | 0.208689071 | 0.761886733 |
| Status_CD8T_PD1_freq | −0.136130266 | 0.196138977 | 0.16750001 |
| ENDO_CD4T | −0.135947881 | 0.193950945 | 0.876392296 |
| Status_CD4T_GZMB_freq | 0.13565994 | 0.190496606 | 0.5000834 |
| Status_DC_GLUT1_freq | −0.13463854 | 0.178243157 | 0.1183337 |
| Status_MAST_HIF1a_freq | 0.13417964 | 0.172737877 | 0.411571678 |
| MONO_CD4T | −0.133674375 | 0.166676355 | 0.497653436 |
| Status_CAF_GLUT1_freq | 0.133358513 | 0.162887048 | 0.962869835 |
| Emask_density_MAST | −0.13234003 | 0.150668597 | 0.58719904 |
| Status_NEUT_Ki67_freq | 0.13228722 | 0.150035055 | 0.420997905 |
| Status_TCELL_MMP9_freq | 0.130834837 | 0.132611231 | 0.404602171 |
| Status_TUMOR_Lumi0l_COX2_freq | −0.130713669 | 0.131157612 | 0.723544789 |
| Status_endo_HIF1a_freq | 0.130129002 | 0.12414353 | 0.212812903 |
| Smask_density_CD8T | 0.129895158 | 0.121338171 | 0.607795875 |
| Allstroma_thick_avg_object_lengths | 0.129700094 | 0.11899804 | 0.598225909 |
| Status_TUMOR_Basal_Ki67_freq | −0.12883034 | 0.108563851 | 0.547287278 |
| MACS_DC | −0.128180783 | 0.100771305 | 0.967185034 |
| Status_TUMOR_CK57low_CD36_freq | 0.127987232 | 0.098449335 | 0.178481105 |
| Status_CD4T_PD1_freq | −0.127198336 | 0.088985167 | 0.729859638 |
| Status_TCELL_HIF1a_freq | 0.126314381 | 0.078380616 | 0.704879353 |
| Status_TUMOR_CK57low_GLUT1_freq | −0.12601945 | 0.074842418 | 0.23730932 |
| Status_CD4T_IDO1_freq | −0.125336846 | 0.066653406 | 0.541813124 |
| Status_fibroblast_HLADR_freq | 0.125284231 | 0.066022209 | 0.891589148 |
| Status_CAF_pS6_freq | 0.124389692 | 0.055290678 | 0.778307816 |
| Smask_lineagefreq_TCELL | 0.123874549 | 0.04911065 | 0.766566468 |
| Status_TUMOR_CK57low_ER_freq | −0.123745752 | 0.047565517 | 0.128145945 |
| Neighborhood_frequency_clust1 | 0.122561478 | 0.033358115 | 0.113909966 |
| Status_BCELL_MMP9_freq | 0.121309772 | 0.018341757 | 0.71968997 |
| Status_TUMOR_Basal_HER2intense_freq | 0.120467405 | 0.008236125 | 0.24275596 |
| Status_TCELL_CD36_freq | 0.119721689 | −0.000710016 | 0.642570346 |
| FIBROBLAST_CD4T | −0.118911342 | −0.010431519 | 0.268246867 |
| ENDO_NEUT | 0.118082558 | −0.020374197 | 0.48334449 |
| Status_CAF_CD44_freq | 0.117015376 | −0.033176875 | 0.461128021 |
| Status_TUMOR_CK57low_pS6_freq | 0.116727231 | −0.036633666 | 0.668572181 |
| Neighborhood_frequency_clust4 | 0.115964962 | −0.045778393 | 0.967907668 |
| Status_TUMOR_Lumi0l_MMP9_freq | 0.115911858 | −0.046415468 | 0.080685929 |
| Status_MYOFIBRO_pS6_freq | −0.114972165 | −0.057688689 | 0.635093614 |
| Status_CAF_MMP9_freq | 0.114439141 | −0.064083224 | 0.817552421 |
| DC_BCELL | 0.113919122 | −0.070321746 | 0.856570855 |
| MAST_ENDO | 0.113224262 | −0.078657782 | 0.105813217 |
| Status_TUMOR_CK57low_AR_freq | −0.112582644 | −0.086355085 | 0.583847414 |
| Wholeimage_lineagefreq_TUMOR_Luminal | 0.112175754 | −0.091236435 | 0.478534532 |
| Status_endo_PDL1_freq | −0.111921063 | −0.094291883 | 0.399701932 |
| Status_tumor_Ki67_freq | 0.111888068 | −0.094687716 | 0.848685087 |
| Status_MAST_pS6_freq | −0.111256411 | −0.102265525 | 0.17354745 |
| periepithelial_fiber_density | 0.110173675 | −0.115254796 | 0.841575294 |
| Status_TUMOR_EMT_GLUT1_freq | −0.108543783 | −0.134808144 | 0.610893611 |
| MAST_TCELL | −0.108293155 | −0.13781486 | 0.92525085 |
| Neighborhood_frequency_clust5 | 0.107917384 | −0.142322871 | 0.593553203 |
| Status_tumor_HER2_freq | −0.107615571 | −0.14594364 | 0.956513543 |
| Status_TUMOR_EMT_HER2intense_freq | 0.107550561 | −0.14672354 | 0.734695851 |
| Status_TUMOR_Basal_IDO1_freq | −0.107405274 | −0.148466513 | 0.888006249 |
| Emask_density_total_tumor | 0.106563558 | −0.158564335 | 0.501380045 |
| FIBROBLAST_TCELL | −0.10618108 | −0.163152813 | 0.852803022 |
| MONO_MONODC | −0.10617483 | −0.1632278 | 0.18599941 |
| FIBROBLAST_MONODC | −0.105961892 | −0.165782345 | 0.664102552 |
| Status_NORMFIBRO_MMP9_freq | −0.105348885 | −0.173136419 | 0.076260109 |
| Emask_density_MONO | −0.105228004 | −0.174586598 | 0.232680877 |
| Status_MYOFIBRO_HLADR_freq | −0.104710412 | −0.180796 | 0.29184309 |
| Status_TUMOR_EMT_IDO1_freq | −0.104125633 | −0.187811422 | 0.970397439 |
| Status_NEUT_CD36_freq | 0.103893244 | −0.190599321 | 0.564499538 |
| Status_MONODC_CD36_freq | 0.103542821 | −0.194803249 | 0.156602732 |
| Status_MONO_GLUT1_freq | 0.102139811 | −0.211634758 | 0.721878407 |
| FIBROBLAST_CD8T | −0.101915513 | −0.2143256 | 0.941553496 |
| Smask_lineagefreq_CD4T | 0.101354537 | −0.221055468 | 0.934369807 |
| Status_MACS_IDO1_freq | −0.099277938 | −0.245967826 | 0.861196436 |
| TCELL_NEUT | −0.099192109 | −0.246997496 | 0.919024664 |
| Wholeimage_lineagefreq_MYOFIBRO | −0.095119061 | −0.295860674 | 0.398121111 |
| FIBROBLAST_ENDO | −0.093517182 | −0.315077962 | 0.855811818 |
| Smask_density_MYOFIBRO | −0.093422335 | −0.316215812 | 0.662764603 |
| APC_DC | −0.09332416 | −0.317393594 | 0.234817652 |
| Status_CD8T_IDO1_freq | 0.092933012 | −0.322086085 | 0.381289666 |
| Status_immune_PDL1_freq | −0.092864611 | −0.322906667 | 0.062220561 |
| Status_MAST_GZMB_freq | 0.092367901 | −0.328865559 | 0.699234332 |
| Emask_lineagefreq_CD8T | −0.092294259 | −0.329749013 | 0.795653597 |
| Status_fibroblast_GLUT1_freq | 0.091339398 | −0.341204216 | 0.799159997 |
| Status_CD8T_COX2_freq | −0.091262758 | −0.342123642 | 0.116697285 |
| Status_TUMOR_EMT_Ki67_freq | −0.091115605 | −0.343888994 | 0.644182049 |
| Emask_lineagefreq_MAST | 0.090313238 | −0.353514759 | 0.831747424 |
| MAST_DC | 0.09009199 | −0.356169005 | 0.069834669 |
| Status_FIBRO_VIMonly_Ki67_freq | 0.090084537 | −0.35625842 | 0.808410694 |
| Status_TUMOR_CK57low_HER2_freq | −0.088199705 | −0.378870208 | 0.941730731 |
| Status_BCELL_GLUT1_freq | 0.087722784 | −0.384591697 | 0.600610365 |
| Status_MACS_MMP9_freq | 0.0869558 | −0.393792974 | 0.96631031 |
| Status_NORMFIBRO_HLADR_freq | 0.08659602 | −0.398109159 | 0.603935486 |
| Status_TCELL_pS6_freq | −0.08658021 | −0.398298828 | 0.768672945 |
| Status_TUMOR_CK57low_MMP9_freq | 0.084285699 | −0.425825409 | 0.77014343 |
| MAST_CD4T | −0.084088001 | −0.428197139 | 0.217569285 |
| Status_CD4T_pS6_freq | −0.083957973 | −0.429757051 | 0.158997663 |
| Status_APC_MMP9_freq | 0.083473003 | −0.435575093 | 0.865441053 |
| Status_MONO_pS6_freq | 0.083296413 | −0.43769359 | 0.570908887 |
| FIBROBLAST_DC | −0.081597826 | −0.458071059 | 0.380918901 |
| Status_immune_pS6_freq | 0.079525685 | −0.482929937 | 0.799184185 |
| MONO_MACS | 0.079217547 | −0.486626576 | 0.624935562 |
| Emask_lineagefreq_MONO | 0.078815967 | −0.491444223 | 0.649315864 |
| MACS_FIBROBLAST | 0.078329524 | −0.497279933 | 0.269508707 |
| Status_immune_CD36_freq | −0.078083589 | −0.50023035 | 0.949285785 |
| Status_FIBRO_VIMonly_pS6_freq | −0.07790731 | −0.502345115 | 0.483619777 |
| Status_NEUT_pS6_freq | 0.07728842 | −0.509769758 | 0.38914107 |
| Status_DC_Ki67_freq | 0.075636472 | −0.529587703 | 0.246806061 |
| MONO_NEUT | −0.075412765 | −0.532271456 | 0.746078337 |
| Status_TUMOR_Lumi0l_GLUT1_freq | 0.074341671 | −0.545121063 | 0.643032843 |
| Status_MONODC_Ki67_freq | −0.073683745 | −0.553014016 | 0.82280025 |
| Status_TUMOR_EMT_CD44_freq | 0.073158591 | −0.559314139 | 0.206621501 |
| Status_MAST_CD36_freq | −0.073068089 | −0.56039986 | 0.281478586 |
| Status_CD4T_Ki67_freq | 0.072745242 | −0.564272969 | 0.040817582 |
| Status_MACS_CD36_freq | −0.07243934 | −0.567942784 | 0.096135699 |
| Status_fibroblast_IDO1_freq | 0.072385632 | −0.568587098 | 0.600950607 |
| Status_MYOFIBRO_CD44_freq | −0.070538098 | −0.590751432 | 0.344392369 |
| Status_endo_SMA_freq | −0.070422479 | −0.592138479 | 0.335178826 |
| Status_TCELL_Ki67_freq | 0.068804806 | −0.611545237 | 0.420997905 |
| Status_immune_MMP9_freq | 0.067597721 | −0.6260263 | 0.978235627 |
| APC_NEUT | −0.06734357 | −0.629075267 | 0.391358388 |
| Status_DC_IDO1_freq | 0.067058242 | −0.632498273 | 0.411446874 |
| distalstroma_area_normalized_intensity | 0.066948676 | −0.633812696 | 0.841575294 |
| Status_MACS_Ki67_freq | −0.066785486 | −0.63577045 | 0.867625447 |
| Status_DC_pS6_freq | −0.066481834 | −0.639413276 | 0.155852441 |
| Smask_lineagefreq_MONODC | 0.066031038 | −0.644821336 | 0.744652925 |
| MACS_CD8T | −0.065781551 | −0.647814366 | 0.321959001 |
| Status_tumor_GLUT1_freq | 0.064309625 | −0.665472634 | 0.913182778 |
| Status_MONODC_MMP9_freq | 0.06072197 | −0.708512705 | 0.558942486 |
| Status_BCELL_IDO1_freq | 0.058250592 | −0.738161122 | 0.84002211 |
| DC_TCELL | 0.057818788 | −0.743341341 | 0.182007238 |
| MAST_CD8T | −0.056495455 | −0.759216993 | 0.810917801 |
| Emask_density_BCELL | −0.055557553 | −0.770468735 | 0.983500039 |
| Status_TUMOR_Basal_pS6_freq | 0.055169607 | −0.77512281 | 0.790016733 |
| DC_MONODC | 0.054845088 | −0.779015968 | 0.079555631 |
| Status_fibroblast_pS6_freq | −0.053804777 | −0.791496288 | 0.604544775 |
| MONODC_CD8T | −0.053445263 | −0.795809267 | 0.40950286 |
| ENDO_MONODC | 0.053267521 | −0.79794159 | 0.514798636 |
| Emask_lineagefreq_APC | 0.053243403 | −0.798230925 | 0.743514717 |
| Epiedge_endo_dist | −0.053161119 | −0.799218063 | 0.054091172 |
| Status_APC_Ki67_freq | −0.052780235 | −0.803787421 | 0.652801943 |
| Status_MONODC_IDO1_freq | 0.052538034 | −0.806693032 | 0.739824633 |
| Status_CD8T_pS6_freq | 0.051349889 | −0.820946864 | 0.923092533 |
| Status_NEUT_IDO1_freq | −0.051248503 | −0.822163173 | 0.625537052 |
| Status_TUMOR_Basal_CD44_freq | −0.051209502 | −0.82263105 | 0.588743983 |
| Status_TUMOR_EMT_pS6_freq | 0.049868592 | −0.83871757 | 0.934824693 |
| APC_MONODC | 0.049449399 | −0.843746504 | 0.443151115 |
| Smask_density_DC | 0.049258259 | −0.846039549 | 0.542170858 |
| MONODC_CD4T | 0.049235334 | −0.846314582 | 0.235945205 |
| Status_fibroblast_MMP9_freq | −0.049233754 | −0.846333532 | 0.151034867 |
| Status_TUMOR_Lumi0l_Ki67_freq | 0.048967381 | −0.849529139 | 0.695303822 |
| Status_NORMFIBRO_CD44_freq | 0.048634294 | −0.853525086 | 0.753195292 |
| Status_TUMOR_CK57low_ | 0.046151114 | −0.883315077 | 0.69300131 |
| HER2intense_freq | | | |
| Smask_lineagefreq_APC | 0.046067651 | −0.884316361 | 0.913169475 |
| APC_CD4T | −0.046063645 | −0.884364422 | 0.2245133 |
| Status_TUMOR_Basal_AR_freq | −0.045745818 | −0.888177304 | 0.881295007 |
| Status_fibroblast_PDL1_freq | −0.045378376 | −0.892585399 | 0.816323941 |
| Smask_density_total_endo | 0.045297243 | −0.893558726 | 0.942058189 |
| Status_TCELL_GZMB_freq | 0.043727583 | −0.912389488 | 0.57270236 |
| Status_immune_GLUT1_freq | 0.043132778 | −0.919525186 | 0.450782112 |
| ENDO_BCELL | −0.042725785 | −0.924407768 | 0.373790696 |
| MACS_CD4T | −0.042693615 | −0.924793704 | 0.985029113 |
| Status_BCELL_PDL1_freq | 0.042482217 | −0.927329789 | 0.667409515 |
| Smask_density_FIBROvimonly | 0.041391629 | −0.940413252 | 0.785189823 |
| Emask_lineagefreq_BECLL | −0.041010797 | −0.944981991 | 0.983500039 |
| MACS_ENDO | 0.040636272 | −0.949475061 | 0.531504972 |
| Status_tumor_ER_freq | −0.040461238 | −0.951574885 | 0.418494078 |
| BCELL_NEUT | 0.039431682 | −0.963926179 | 0.703461179 |
| Status_CD4T_CD36_freq | −0.03883336 | −0.971104078 | 0.370939408 |
| APC_MACS | −0.038802097 | −0.971479125 | 0.664634178 |
| Emask_density_DC | −0.038331121 | −0.977129297 | 0.450045991 |
| tumor_shannon | 0.038234199 | −0.978292037 | 0.971009966 |
| Status_MAST_COX2_freq | 0.036983838 | −0.99329226 | 0.947358111 |
| MONO_TCELL | 0.036975542 | −0.993391782 | 0.852310516 |
| Status_TUMOR_CK57low_Ki67_freq | −0.03668956 | −0.996822626 | 0.229219184 |
| Status_APC_GLUT1_freq | −0.036472141 | −0.999430938 | 0.629641462 |
| ENDO_TCELL | −0.03626335 | −1.001935741 | 0.926085025 |
| Status_MONODC_PDL1_freq | −0.03435933 | −1.024777727 | 0.411571678 |
| CD8T_NEUT | −0.033460268 | −1.035563514 | 0.268940143 |
| Status_CAF_HLADR_freq | 0.033331479 | −1.037108564 | 0.984959393 |
| Status_immune_IDO1_freq | −0.033123789 | −1.039600158 | 0.970955439 |
| MAST_FIBROBLAST | 0.033121033 | −1.039633219 | 0.716277318 |
| Status_CD8T_GZMB_freq | 0.032885607 | −1.042457565 | 0.667409515 |
| Status_NORMFIBRO_pS6_freq | −0.03253135 | −1.046707474 | 0.977053153 |
| Status_tumor_MMP9_freq | −0.031632585 | −1.0574897 | 0.029813319 |
| Status_TUMOR_Basal_ER_freq | −0.031550689 | −1.058472188 | 0.32167453 |
| Status_TUMOR_Lumi0l_HER2_freq | −0.03132876 | −1.061134602 | 0.682085571 |
| Status_MAST_IDO1_freq | 0.031138584 | −1.06341609 | 0.758579829 |
| MONO_DC | 0.030668194 | −1.069059222 | 0.179811555 |
| Status_CD8T_Ki67_freq | 0.030667296 | −1.069070002 | 0.771924032 |
| Status_TCELL_COX2_freq | 0.029808385 | −1.079374106 | 0.983500039 |
| Status_TUMOR_Basal_COX2_freq | −0.029179352 | −1.086920437 | 0.276019347 |
| Status_MAST_PDL1_freq | −0.028848295 | −1.090892031 | 0.862635228 |
| Status_BCELL_CD36_freq | −0.028331847 | −1.097087713 | 0.699979801 |
| Status_APC_COX2_freq | 0.027594212 | −1.105936911 | 0.670836899 |
| Status_MONO_CD36_freq | 0.027481683 | −1.107286889 | 0.144986474 |
| Status_APC_IDO1_freq | 0.027420672 | −1.108018814 | 0.975383798 |
| Status_TUMOR_EMT_CD36_freq | −0.027017899 | −1.112850767 | 0.848556165 |
| Status_TUMOR_EMT_MMP9_freq | −0.026795056 | −1.11552415 | 0.288571096 |
| mcShannon | −0.026313776 | −1.121297933 | 0.870095423 |
| Status_APC_pS6_freq | −0.025637707 | −1.129408539 | 0.517153658 |
| Status_TUMOR_LumiOl_CD44_freq | −0.025570637 | −1.130213151 | 0.949288901 |
| Status_TUMOR_EMT_HER2_freq | −0.024734549 | −1.140243465 | 1 |
| Status_MONODC_pS6_freq | −0.023686663 | −1.152814647 | 0.460932009 |
| Smask_density_total_fibroblast | 0.023123285 | −1.15957333 | 0.927608566 |
| Emask_lineagefreq_CD4T | −0.023056827 | −1.160370613 | 0.896027335 |
| Status_immune_Ki67_freq | −0.022593443 | −1.165929695 | 0.790023176 |
| Status_CD8T_CD36_freq | 0.02241591 | −1.168059508 | 0.797010057 |
| Status_NORMFIBRO_IDO1_freq | 0.022114692 | −1.171673129 | 0.607713155 |
| Status_tumor_CD44_freq | −0.021964831 | −1.173470976 | 0.841575294 |
| MAST_APC | −0.021672574 | −1.176977101 | 0.942040389 |
| Wholeimage_density_log2fc_myeloid_to_ | −0.021512803 | −1.178893821 | 0.317558965 |
| lymphoid | | | |
| Smask_lineagefreq_CD8T | 0.020999945 | −1.18504644 | 0.707096908 |
| DC_CD4T | 0.020977057 | −1.185321015 | 0.636546806 |
| MONODC_NEUT | 0.0207928 | −1.187531495 | 0.579800435 |
| MONO_ENDO | 0.020390259 | −1.192360664 | 0.80619035 |
| Status_CD4T_GLUT1_freq | −0.020161267 | −1.195107811 | 0.342927212 |
| Emask_density_CD4T | −0.018648203 | −1.213259607 | 0.663137517 |
| Wholeimage_lineagefreq_FIBROvimonly | −0.018445986 | −1.215685544 | 0.956526908 |
| Status_MONODC_GLUT1_freq | 0.018282443 | −1.217647528 | 0.766166702 |
| Status_tumor_IDO1_freq | −0.017850671 | −1.222827374 | 0.770271092 |
| Status_TUMOR_EMT_ER_freq | −0.017329864 | −1.229075347 | 0.506785795 |
| Status_TUMOR_EMT_COX2_freq | 0.01609026 | −1.243946518 | 0.87327132 |
| Status_MAST_Ki67_freq | 0.015291406 | −1.253530131 | 0.380392091 |
| Emask_lineagefreq_MONODC | −0.014974391 | −1.257333279 | 0.442387605 |
| Status_tumor_CD36_freq | 0.0147338 | −1.26021958 | 0.215673432 |
| Status_MONO_Ki67_freq | 0.013876018 | −1.27051014 | 0.510855072 |
| Status_TUMOR_Lumi0l_IDO1_freq | 0.01381527 | −1.271238916 | 0.411687104 |
| Status_MAST_GLUT1_freq | −0.013250915 | −1.278009319 | 0.217292436 |
| fibro_shannon | −0.012552348 | −1.286389825 | 0.757394743 |
| Status_CD4T_COX2_freq | 0.011584856 | −1.297996558 | 0.307521804 |
| Smask_lineagefreq_MONO | 0.011388439 | −1.300352917 | 0.200095852 |
| Smask_density_MAST | −0.010742763 | −1.308098904 | 0.105813217 |
| MONODC_TCELL | 0.010643174 | −1.309293636 | 0.511726455 |
| Status_MAST_MMP9_freq | 0.00974308 | −1.320091814 | 0.740342835 |
| Status_immune_COX2_freq | 0.009635165 | −1.321386434 | 0.956333247 |
| Smask_lineagefreq_DC | −0.009340872 | −1.324916984 | 0.42368774 |
| Status_tumor_AR_freq | 0.007711161 | −1.344468156 | 0.853988004 |
| MONO_CD8T | −0.007627377 | −1.345473295 | 0.970757114 |
| Status_TUMOR_Lumi0l_ER_freq | −0.007379021 | −1.348452751 | 0.584387885 |
| Allstroma_thick_total_object_densities | 0.005853263 | −1.36675683 | 0.501380045 |
| distalstroma_fiber_density | 0.005661412 | −1.369058405 | 0.548748387 |
| Emask_density_APC | 0.005384021 | −1.37238619 | 0.895926718 |
| Status_MACS_pS6_freq | −0.005106928 | −1.375710399 | 0.482116116 |
| Status_NORMFIBRO_GLUT1_freq | 0.003351184 | −1.39677355 | 0.353199616 |
| Status_TUMOR_Basal_CD36_freq | −0.002510991 | −1.406853103 | 0.034700983 |
| Status_MONO_PDL1_freq | 0.002351144 | −1.408770741 | 0.851958057 |
| Status_TUMOR_Lumi0l_AR_freq | 0.002066134 | −1.41218993 | 0.778350315 |
| Neighborhood_frequency_clust2 | 0.000247149 | −1.434011763 | 0.799193252 |
| Status_TUMOR_Lumi0l_CD36_freq | 0.000212186 | −1.434431215 | 0.755988738 |

TABLE 2
LD1 Correlation Feature Ontology

| pathway | pval | padj | log2err | ES | NES | size | leadingEdge |
|---|---|---|---|---|---|---|---|
| Desmoplasia | 0.031394368 | 0.069067609 | 0.321775918 | 0.414238606 | 1.559291583 | 36 | Wholeimage_lineagefreq_CAF |
| and | | | | | | | Status_FIBRO_VIMonly_MMP9_freq |
| ECM | | | | | | | Smask_density_CAF |
| remodeling | | | | | | | Status_MONO_MMP9_freq |
| | | | | | | | midstroma_thick_avg_object_areas |
| | | | | | | | midstroma_fiber_density |
| | | | | | | | midstroma_area_normalized_intensity |
| | | | | | | | Allstroma_avg_branch_count |
| | | | | | | | distalstroma_thick_avg_object_areas |
| | | | | | | | periepithelial_area_normalized_intensity |
| | | | | | | | Status_CD8T_MMP9_freq |
| | | | | | | | Status_NEUT_MMP9_freq |
| | | | | | | | periepithelial_thick_avg_object_areas |
| | | | | | | | Status_TCELL_MMP9_freq |
| | | | | | | | Allstroma_thick_avg_object_lengths |
| | | | | | | | Status_BCELL_MMP9_freq |
| | | | | | | | Status_TUMOR_Lumi0l_MMP9_freq |
| | | | | | | | Status_CAF_MMP9_freq |
| | | | | | | | periepithelial_fiber_density |
| | | | | | | | Status_MACS_MMP9_freq |
| | | | | | | | Status_TUMOR_CK57low_MMP9_freq |
| | | | | | | | Status_APC_MMP9_freq |
| | | | | | | | Status_immune_MMP9_freq |
| | | | | | | | distalstroma_area_normalized_intensity |
| | | | | | | | Status_MONODC_MMP9_freq |
| Immune: | 0.009950974 | 0.027365179 | 0.380730401 | −0.444292373 | −1.62837538 | 32 | Status_immune_PD1_freq |
| immunoregulation | | | | | | | Status_APC_PDL1_freq |
| | | | | | | | Status_TCELL_IDO1_freq |
| | | | | | | | Status_MACS_PDL1_freq |
| | | | | | | | Status_BCELL_PD1_freq |
| | | | | | | | Status_TCELL_PD1_freq |
| | | | | | | | Status_DC_PDL1_freq |
| | | | | | | | Status_CD8T_PD1_freq |
| | | | | | | | Status_CD4T_PD1_freq |
| | | | | | | | Status_CD4T_IDO1_freq |
| | | | | | | | Status_MACS_IDO1_freq |
| | | | | | | | Status_immune_PDL1_freq |
| | | | | | | | Status_CD8T_COX2_freq |
| Lipid | 0.04761962 | 0.087302637 | 0.321775918 | −0.486301633 | −1.549549771 | 18 | Status_APC_CD36_freq |
| metabolism | | | | | | | Status_DC_CD36_freq |
| | | | | | | | Status_endo_CD36_freq |
| Lymphoid: | 0.743027888 | 0.908145197 | 0.059221919 | 0.341911656 | 0.788520562 | 7 | Status_BCELL_pS6_freq |
| growth/ | | | | | | | Status_CD4T_Ki67_freq |
| proliferation | | | | | | | Status_TCELL_Ki67_freq |
| | | | | | | | Status_CD8T_pS6_freq |
| | | | | | | | Status_CD8T_Ki67_freq |
| Myeloid: | 0.893223819 | 0.936633663 | 0.052057003 | −0.233595801 | −0.684908012 | 14 | Status_MAST_pS6_freq |
| growth/ | | | | | | | Status_MONODC_Ki67_freq |
| proliferation | | | | | | | Status_MACS_Ki67_freq |
| | | | | | | | Status_DC_pS6_freq |
| | | | | | | | Status_APC_Ki67_freq |
| | | | | | | | Status_APC_pS6_freq |
| | | | | | | | Status_MONODC_pS6_freq |
| | | | | | | | Status_MACS_pS6_freq |
| | | | | | | | Status_MONO_Ki67_freq |
| | | | | | | | Status_MAST_Ki67_freq |
| | | | | | | | Status_DC_Ki67_freq |
| | | | | | | | Status_NEUT_pS6_freq |
| | | | | | | | Status_MONO_pS6_freq |
| | | | | | | | Status_NEUT_Ki67_freq |
| Immune | 0.002552215 | 0.009358123 | 0.431707696 | 0.665806063 | 1.808546369 | 12 | Smask_density_NEUT |
| density | | | | | | | Smask_density_APC |
| in stroma | | | | | | | Smask_density_TCELL |
| | | | | | | | Smask_density_immune |
| | | | | | | | Smask_density_CD4T |
| | | | | | | | Smask_density_BCELL |
| | | | | | | | Smask_density_MONO |
| | | | | | | | Smask_density_MONODC |
| | | | | | | | Smask_density_CD8T |
| Stroma: | 0.1021611 | 0.160538872 | 0.195789002 | 0.511860801 | 1.39038084 | 12 | Status_NORMFIBRO_Ki67_freq |
| growth/ | | | | | | | Status_endo_Ki67_freq |
| proliferation | | | | | | | Status_CAF_Ki67_freq |
| | | | | | | | Status_fibroblast_Ki67_freq |
| | | | | | | | Status_MYOFIBRO_Ki67_freq |
| Tumor: | 0.936633663 | 0.936633663 | 0.048214971 | 0.198135334 | 0.643103757 | 21 | Status_TUMOR_Basal_HER2_freq |
| ER/AR/HER2 | | | | | | | Status_TUMOR_Lumi0l_HER2intense_freq |
| expression | | | | | | | Status_tumor_HER2intense_freq |
| | | | | | | | Status_tumor_HER2_log2fc_int_vs_pos |
| | | | | | | | Status_TUMOR_Basal_HER2intense_freq |
| | | | | | | | Status_TUMOR_EMT_HER2intense_freq |
| Tumor: | 5.67E−05 | 0.000312095 | 0.557332239 | −0.740639391 | −2.214914484 | 15 | Status_TUMOR_Basal_PDL1_freq |
| immunoregulation | | | | | | | Status_TUMOR_CK57low_PDL1_freq |
| | | | | | | | Status_tumor_COX2_freq |
| | | | | | | | Status_TUMOR_CK57low_IDO1_freq |
| | | | | | | | Status_TUMOR_Lumi0l_PDL1_freq |
| | | | | | | | Status_tumor_PDL1_freq |
| | | | | | | | Status_TUMOR_EMT_PDL1_freq |
| | | | | | | | Status_TUMOR_CK57low_COX2_freq |
| | | | | | | | Status_TUMOR_Lumi0l_COX2_freq |
| | | | | | | | Status_TUMOR_Basal_IDO1_freq |
| | | | | | | | Status_TUMOR_EMT_IDO1_freq |
| Tumor: | 0.554474708 | 0.762402724 | 0.072357085 | 0.359674142 | 0.92523568 | 10 | Status_tumor_pS6_freq |
| growth/ | | | | | | | Status_TUMOR_Lumi0l_pS6_freq |
| proliferation | | | | | | | Status_TUMOR_CK57low_pS6_freq |
| | | | | | | | Status_tumor_Ki67_freq |
| | | | | | | | Status_TUMOR_Basal_pS6_freq |
| | | | | | | | Status_TUMOR_EMT_pS6_freq |
| | | | | | | | Status_TUMOR_Lumi0l_Ki67_freq |
| Hypoxia | 2.67E−06 | 2.94E−05 | 0.62725674 | 0.596413329 | 2.343828288 | 42 | Status_TUMOR_EMT_HIF1a_freq |
| and | | | | | | | Status_MONO_HIF1a_freq |
| Glycolysis | | | | | | | Status_immune_HIF1a_freq |
| | | | | | | | Status_TCELL_GLUT1_freq |
| | | | | | | | Status_MACS_HIF1a_freq |
| | | | | | | | Status_TUMOR_CK57low_HIF1a_freq |
| | | | | | | | Status_tumor_HIF1a_freq |
| | | | | | | | Status_APC_HIF1a_freq |
| | | | | | | | Status_TUMOR_Basal_HIF1a_freq |
| | | | | | | | Status_endo_GLUT1_freq |
| | | | | | | | Status_TUMOR_Lumi0l_HIF1a_freq |
| | | | | | | | Status_CD8T_HIF1a_freq |
| | | | | | | | Status_DC_HIF1a_freq |
| | | | | | | | Status_CD4T_HIF1a_freq |
| | | | | | | | Status_NEUT_HIF1a_freq |
| | | | | | | | Status_FIBRO_VIMonly_GLUT1_freq |
| | | | | | | | Status_MYOFIBRO_GLUT1_freq |
| | | | | | | | Status_fibroblast_HIF1a_freq |
| | | | | | | | Status_CD8T_GLUT1_freq |
| | | | | | | | Status_NEUT_GLUT1_freq |
| | | | | | | | Status_BCELL_HIF1a_freq |
| | | | | | | | Status_MONODC_HIF1a_freq |
| | | | | | | | Status_MAST_HIF1a_freq |
| | | | | | | | Status_CAF_GLUT1_freq |
| | | | | | | | Status_endo_HIF1a_freq |

### REFERENCES

Afghahi, A., Forgó, E., Mitani, A. A., Desai, M., Varma, S., Seto, T., Rigdon, J., Jensen, K. C., Troxell, M. L., Gomez, S. L., et al. (2015). Chromosomal copy number alterations for associations of ductal carcinoma in situ with invasive breast cancer. Breast Cancer Res. 17, 108.
Aguiar, F. N., Cirqueira, C. S., Bacchi, C. E., and Carvalho, F. M. (2015). Morphologic, molecular and microenvironment factors associated with stromal invasion in breast ductal carcinoma in situ: Role of myoepithelial cells. Breast Dis. 35, 249-252.
Ak, C., A, S., R, G., E, S., A, L., W, P., T, C., F, M.-B., Me, E., and Ne, N. (2018). Multiclonal
Invasion in Breast Tumors Identified by Topographic Single Cell Sequencing (Cell).
Alcazar, C. R. G. D., Huh, S. J., Ekram, M. B., Trinh, A., Liu, L. L., Beca, F., Zi, X., Kwak, M., Bergholtz, H., Su, Y., et al. (2017). Immune Escape in Breast Cancer During In Situ to Invasive Carcinoma Transition. Cancer Discov. 7, 1098-1115.
Anders, S., and Huber, W. (2010). Differential expression analysis for sequence count data. Genome Biol. 11, R106.
Aponte-López, A., Fuentes-Pananá, E. M., Cortes-Muñoz, D., and Muñoz-Cruz, S. (2018). MastCell, the Neglected Member of the Tumor Microenvironment: Role in Breast Cancer.
Barsky, S. H., and Karlin, N. J. (2005). Myoepithelial Cells: Autocrine and Paracrine Suppressors of Breast Cancer Progression. J. Mammary Gland Biol. Neoplasia 10, 249-260.
Barth, P. J., Moll, R., and Ramaswamy, A. (2005). Stromal remodeling and SPARC (secreted protein acid rich in cysteine) expression in invasive ductal carcinomas of the breast. VirchowsArch. 446, 532-536.
Bartova, M., Ondrias, F., Muy-Kheng, T., Kastner, M., Singer, C., and Pohlodek, K. (2014). COX-2,p16 and Ki67 expression in DCIS, microinvasive and early invasive breast carcinoma with extensive intraductal component. Bratisl. Lek. Listy 115, 445-451.
Betsill, W. L., Rosen, P. P., Lieberman, P. H., and Robbins, G. F. (1978). Intraductal carcinoma. Long-term follow-up after treatment by biopsy alone. JAMA 239, 1863-1867.
Buerger, H., Otterbach, F., Simon, R., Poremba, C., Diallo, R., Decker, T., Riethdorf, L., Brinkschmidt, C., Dockhorn-Dworniczak, B., and Boecker, W. (1999). Comparative genomic hybridization of ductal carcinoma in situ of the breast-evidence of multiple genetic pathways. J. Pathol. 187, 396-402.
Cancer Genome Atlas Network (2012). Comprehensive molecular portraits of human breast tumours. Nature 490, 61-70.
Conklin, M. W., Eickhoff, J. C., Riching, K. M., Pehlke, C. A., Eliceiri, K. W., Provenzano, P. P., Friedl, A., and Keely, P. J. (2011). Aligned Collagen Is a Prognostic Signature for Survival in Human Breast Carcinoma. Am. J. Pathol. 178, 1221-1232.
Curtis, C., Shah, S. P., Chin, S.-F., Turashvili, G., Rueda, O. M., Dunning, M. J., Speed, D., Lynch, A. G., Samarajiwa, S., Yuan, Y., et al. (2012). The genomic and transcriptomic architecture of 2,000 breast tumours reveals novel subgroups. Nature 486, 346-352.
Ding, L., Su, Y., Fassl, A., Hinohara, K., Qiu, X., Harper, N. W., Huh, S. J., Bloushtain-Qimron, N., Jovanović, B., Ekram, M., et al. (2019). Perturbed myoepithelial cell differentiation in BRCA mutation carriers and in ductal carcinoma in situ. Nat. Commun. 10, 4182.
Erbas, B., Provenzano, E., Armes, J., and Gertig, D. (2006). The natural history of ductal carcinoma <Emphasis Type=“Boldltalic”>in situ</Emphasis> of the breast: a review. Breast Cancer Res. Treat. 97, 135-144.
Esbona, K., Yi, Y., Saha, S., Yu, M., Doorn, R. R. V., Conklin, M. W., Graham, D. S., Wisinski, K. B., Ponik, S. M., Eliceiri, K. W., et al. (2018). The Presence of Cyclooxygenase 2, Tumor-Associated
Macrophages, and Collagen Alignment as Prognostic Markers for Invasive Breast Carcinoma Patients. Am. J. Pathol. 188, 559-573.
Eusebi, V., Feudale, E., Foschini, M. P., Micheli, A., Conti, A., Riva, C., Di Palma, S., and Rilke, F. (1994). Long-term follow-up of in situ carcinoma of the breast. Semin. Diagn. Pathol. 11, 223-235.
Foley, J. W., Zhu, C., Jolivet, P., Zhu, S. X., Lu, P., Meaney, M. J., and West, R. B. (2019). Gene expression profiling of single cells from archival tissue with laser-capture microdissection and Smart-3SEQ. Genome Res. 29, 1816-1825.
Friedman, G., Levi-Galibov, O., David, E., Bornstein, C., Giladi, A., Dadiani, M., Mayo, A., Halperin, C., Pevsner-Fischer, M., Lavon, H., et al. (2020). Cancer-associated fibroblast compositions change with breast-cancer progression linking S100A4 and PDPN ratios with clinical outcome. BioRxiv 2020.01.12.903039.
Fujii, H., Szumel, R., Marsh, C., Zhou, W., and Gabrielson, E. (1996). Genetic progression, histological grade, and allelic loss in ductal carcinoma in situ of the breast. Cancer Res. 56, 5260-5265.
Greenwald, N.F., Miller, G., Moen, E., Kong, A., Kagel, A., Fullaway, C. C., McIntosh, B. J., Leow, K., Schwartz, M. S., Dougherty, T., et al. (2021). Whole-cell segmentation of tissue images with human-level performance using large-scale data annotation and deep learning. BioRxiv 2021.03.01.431313.
Ibrahim, A. M., Moss, M. A., Gray, Z., Rojo, M. D., Burke, C. M., Schwertfeger, K. L., dos Santos, C. O., and Machado, H. L. (2020). Diverse Macrophage Populations Contribute to the Inflammatory Microenvironment in Premalignant Lesions During Localized Invasion. Front. Oncol. 10.
Jones, J. L., Shaw, J. A., Pringle, J. H., and Walker, R. A. (2003). Primary breast myoepithelial cellsexert an invasion-suppressor effect on breast cancer cells via paracrine down-regulation of MMP expression in fibroblasts and tumour cells. J. Pathol. 201, 562-572.
Keren, L., Bosse, M., Marquez, D., Angoshtari, R., Jain, S., Varma, S., Yang, S.-R., Kurian, A., VanValen, D., West, R., et al. (2018). A Structured Tumor-Immune Microenvironment in Triple Negative Breast Cancer Revealed by Multiplexed Ion Beam Imaging. Cell 174, 1373-1387.e19.
Keren, L., Bosse, M., Steve, T., Risom, T., Vijayaragavan, K., McCaffrey, E., Angoshtari, R., Greenwald, N., Fienberg, H., Wang, J., et al. (2019). MIBI-TOF: A multi-modal multiplexed imaging platform for tissue pathology. Sci. Adv. In Press.
Kim, S. Y., Jung, S.-H., Kim, M. S., Baek, I.-P., Lee, S. H., Kim, T.-M., Chung, Y.-J., and Lee, S. H.
(2015). Genomic differences between pure ductal carcinoma in situ and synchronous ductal carcinoma in situ with invasive breast cancer. Oncotarget 6, 7597-7607.
Korotkevich, G., Sukhov, V., Budin, N., Shpak, B., Artyomov, M. N., and Sergushichev, A. (2021). Fast gene set enrichment analysis. BioRxiv 060012.
Malanchi, I., Santamaria-Martínez, A., Susanto, E., Peng, H., Lehr, H.-A., Delaloye, J.-F., and Huelsken, J. (2012). Interactions between cancer stem cells and their niche govern metastatic colonization. Nature 481, 85-89.
McCaffrey, E. F., Donato, M., Keren, L., Chen, Z., Fitzpatrick, M., Jojic, V., Delmastro, A., Greenwald, N. F., Baranski, A., Graf, W., et al. (2020). Multiplexed imaging of human tuberculosis granulomas uncovers immunoregulatory features conserved across tissue and blood. BioRxiv 2020.06.08.140426.
Moen, E., Bannon, D., Kudo, T., Graf, W., Covert, M., and Van Valen, D. (2019). Deep learning for cellular image analysis. Nat. Methods 16, 1233-1246.
Newburger, D. E., Kashef-Haghighi, D., Weng, Z., Salari, R., Sweeney, R. T., Brunner, A. L., Zhu, S. X., Guo, X., Varma, S., Troxell, M. L., et al. (2013). Genome evolution during progression to breast cancer. Genome Res. 23, 1097-1108.
Page, D. L., Dupont, W. D., Rogers, L. W., and Landenberger, M. (1982). Intraductal carcinoma of the breast: follow-up after biopsy only. Cancer 49, 751-758.
Pelon, F., Bourachot, B., Kieffer, Y., Magagna, I., Mermet-Meillon, F., Bonnet, I., Costa, A., Givel, A.-M., Attieh, Y., Barbazan, J., et al. (2020). Cancer-associated fibroblast heterogeneity in axillary lymph nodes drives metastases in breast cancer through complementary mechanisms. Nat. Commun. 11, 404.
Perez, A. A., Balabram, D., Rocha, R. M., da Silva Souza, A., and Gobbi, H. (2015). Co-Expression of p16, Ki67 and COX-2 Is Associated with Basal Phenotype in High-Grade Ductal Carcinoma InSitu of the Breast. J. Histochem. Cytochem. Off. J. Histochem. Soc. 63, 408-416.
Rakovitch, E., Nofech-Mozes, S., Hanna, W., Narod, S., Thiruchelvam, D., Saskin, R., Spayne, J., Taylor, C., and Paszat, L. (2012). HER2/neu and Ki-67 expression predict non-invasive recurrence following breast-conserving therapy for ductal carcinoma in situ. Br. J. Cancer 106, 1160-1165.
Robin, X., Turck, N., Hainard, A., Tiberti, N., Lisacek, F., Sanchez, J.-C., and Müller, M. (2011). pROC: an open-source package for R and S+to analyze and compare ROC curves. BMC Bioinformatics 12, 77.
Ryser, M. D., Weaver, D. L., Zhao, F., Worni, M., Grimm, L. J., Gulati, R., Etzioni, R., Hyslop, T., Lee, S. J., and Hwang, E. S . (2019). Cancer Outcomes in DCIS Patients Without Locoregional Treatment. JNCI J. Natl. Cancer Inst. 111, 952-960.
Shani, O., Vorobyov, T., Monteran, L., Lavie, D., Cohen, N., Raz, Y., Tsarfaty, G., Avivi, C., Barshack, I., and Erez, N. (2020). Fibroblast-derived IL-33 facilitates breast cancer metastasis by modifying the immune microenvironment and driving type-2 immunity. Cancer Res.
Sirka, O. K., Shamir, E. R., and Ewald, A. J. (2018). Myoepithelial cells are a dynamic barrier to epithelial dissemination. J. Cell Biol. 217, 3368-3381.
Sprague, B. L., Vacek, P. M., Mulrow, S. E., Evans, M. F., Trentham-Dietz, A., Herschorn, S. D., James, T. A., Surachaicharn, N., Keikhosravi, A., Eliceiri, K. W., et al. (2021). Collagen Organization in Relation to Ductal Carcinoma In Situ Pathology and Outcomes. Cancer Epidemiol. Biomark.
Prey. Publ. Am. Assoc. Cancer Res. Cosponsored Am. Soc. Prey. Oncol. 30, 80-88.
Tsai, A. G., Glass, D. R., Juntilla, M., Hartmann, F. J., Oak, J. S., Fernandez-Pol, S., Ohgami, R. S., and Bendall, S. C. (2020). Multiplexed single-cell morphometry for hematopathology diagnostics.
Nat. Med. 26, 408-417.
Valen, D. A. V., Kudo, T., Lane, K. M., Macklin, D. N., Quach, N. T., DeFelice, M. M., Maayan, I., Tanouchi, Y., Ashley, E. A., and Covert, M. W. (2016). Deep Learning Automates the Quantitative Analysis of Individual Cells in Live-Cell Imaging Experiments. PLOS Comput. Biol. 12, e1005177.
Van Gassen, S., Callebaut, B., Van Heiden, M. J., Lambrecht, B. N., Demeester, P., Dhaene, T., and Saeys, Y. (2015). FlowSOM: Using self-organizing maps for visualization and interpretation of cytometry data. Cytom. Part J. Int. Soc. Anal. Cytol. 87, 636-645.
Wright, M. N., and Ziegler, A. (2017). ranger: A Fast Implementation of Random Forests for High Dimensional Data in C++ and R. J. Stat. Softw. 77, 1-17.
Yang, M., Li, Z., Ren, M., Li, S., Zhang, L., Zhang, X., and Liu, F. (2018). Stromal Infiltration of Tumor-Associated Macrophages Conferring Poor Prognosis of Patients with Basal-Like Breast Carcinoma. J. Cancer 9, 2308-2316.
Zhou, J., Wang, X.-H., Zhao, Y.-X., Chen, C., Xu, X.-Y., sun, Q., Wu, H.-Y., Chen, M., Sang, J.-F., Su,
L., et al. (2018). Cancer-Associated Fibroblasts Correlate with Tumor-Associated Macrophages Infiltration and Lymphatic Metastasis in Triple Negative Breast Cancer Patients. J. Cancer 9, 4635-4641.

1. A method of classifying a ductal carcinoma in situ (DCIS) lesion as indolent, or invasive recurrent, the method comprising:
obtaining a sample of the DCIS lesion;
analyzing the sample for ductal myoepithelium features; and
classifying the DCIS lesion, wherein a DCIS sample comprising myoepitheliem characterized as thin, discontinuous, low E-cadherin (ECAD) expressing myoepithelium, relative to a normal control, is classified as indolent and a DCIS sample comprising continuous myoepithelium with high ECAD expression is classified as invasive recurrent.

2. The method of claim 1, further comprising treating the DCIS lesion in accordance with the classification.

3. The method of claim 1, wherein the analyzing comprises contacting the sample with one or a panel of antibodies comprising least an antibody specific for ECAD.

4. The method of claim 1, wherein the analyzing comprises performing multiplexed ion beam imaging by time of flight (MIBI-TOF) analysis of the lesion sample.

5. The method of claim 4, wherein analyzing the sample comprises analysis of features extracted from MIBI-TOF data, including one or more of phenotypic, functional, spatial, and morphologic features.

6. A method of classifying a ductal carcinoma in situ (DCIS) lesion as indolent; or invasive recurrent, the method comprising:
obtaining a sample of the DCIS lesion;
contacting the sample of the DCIS lesion with a panel of antibodies comprising antibodies specific for one or more markers selected from Tryptase, CK7, VIM, CD44, CK5, PanCK, HIF1A, CD45, AR, HLADR/DP/DQ, GLUT1, ECAD, CD20, MMP9, FAP, CD11c, HER2, CD3, CD8, CD36, MPO, CD68, pS6, Granzyme B, P63, Ki67, IDO1, CD31, PD1, CD14, CD4, Collagen 1, SMA, COX2, Histone H3, ER, and PDL1; and
extracting one or more of phenotypic, functional, spatial, and morphologic features from the DCIS lesion;
classifying the DCIS lesion with a random forest classifier implemented on a computer system, trained on patients with known clinical outcomes.

7. The method of claim 6, further comprising treating the DCIS lesion in accordance with the classification.

8. The method of claim 6, wherein the panel comprises at least 5, at least 10, at least 15, at least 20, at least 25, at least 30, at least 35 or all of the markers.

9. The method of claim 6, comprising MIBI-TOF analysis of the lesion following contacting with the panel of antibodies to extract a plurality of features.

10. The method of claim 9, wherein the features for classification comprise one or more of: myoepithelial E-cadherin, antigen presenting cells (APC) near endothelium, periductal immune cells, ER+luminal tumor cells, ER+tumor cells, myoepithelial CKS, tumor-myoepithelial neighborhood, APC near fibroblast, CD8+T cells near double negative T cells (dnT), myoepithelial continuity, CD4+T cells near dnT, stromal mast cells, PDL1+CK5/7-low tumor cells, tumor-dominate neighborhood, B cell near dnT, nacrophage near mast cells, CD8+T cells near mast cells, variation in collagen fiber orientation, periductal APCs, and PD1+immune cells.

11. The method of claim 9, wherein the features for classification comprise each of:
myoepithelial E-cadherin, antigen presenting cells (APC) near endothelium, periductal immune cells, ER+luminal tumor cells, ER+tumor cells, myoepithelial CK5, tumor-myoepithelial neighborhood, APC near fibroblast, CD8+T cells near double negative T cells (dnT), myoepithelial continuity, CD4+T cells near dnT, stromal mast cells, PDL1+CK5/7-low tumor cells, tumor-dominate neighborhood, B cell near dnT, nacrophage near mast cells, CD8+T cells near mast cells, variation in collagen fiber orientation, periductal APCs, and PD1+immune cells.

12. The method of claim 1, comprising determining the presence of ECAD⁺ myoepithelial expression as indicative of a recurrent phenotype.

13. The method of claim 6, comprising determining stromal density of PanCK⁺VIM⁺ cells as indicative of a recurrent phenotype.

14. The method of claim 6, wherein the features comprise metrics related to the phenotype of myoepithelium, the structure of collagen fibers in the extracellular matrix, and the spatial distribution of multiple immune cell subsets.

15. The method of claim 6, wherein the features comprise spatial metrics describing cell densities, cell neighborhoods, pairwise cell distances, collagen structure, and multiplexed subcellular features.
